(19) United States
(12) Patent Application Publication
(10) Pub. No.: US 2023/0326586 A1
(43) Pub. Date: Oct. 12, 2023

(54) SYSTEMS AND METHODS FOR FACILITATING REMOTE TESTING USING COMPUTER VISION, GIG-ECONOMY QUALITY ASSURANCE SYSTEM FOR DATA LABELING AND DISCRETE TASK COMPLETION AND PROCTOR MANAGEMENT

(71) Applicant: EMED LABS, LLC, Miami, FL (US)

(72) Inventors: Zachary Carl Nienstedt, Wilton Manors, FL (US); John Andrew Sands, Weston, FL (US); Michael W. Ferro, Palm Beach, FL (US); Sam Miller, Hollywood, FL (US); Randall Eugene Hand, Parkland, FL (US); James Thomas Heising, Richland, WA (US)

(21) Appl. No.: 18/194,210

(22) Filed: Mar. 31, 2023

Related U.S. Application Data
(60) Provisional application No. 63/363,433, filed on Apr. 22, 2022.
(60) Provisional application No. 63/336,621, filed on Apr. 29, 2022.
(60) Provisional application No. 63/364,671, filed on May 13, 2022.
(60) Provisional application No. 63/362,576, filed on Apr. 6, 2022.
(60) Provisional application No. 63/363,358, filed on Apr. 21, 2022.

(51) Int. Cl. G16H 40/20; G06Q 10/0631

(52) U.S. Cl. CPC G16H 40/20; G06Q 10/063112; G06K 7/1417

(57) ABSTRACT

At-home diagnostic tests kits may be used by individuals to self-administer a diagnostic test. The diagnostic test kit can include one or more machine-readable codes on the surface of the test kit which may indicate information about the diagnostic test, such as whether a proctor is required during administration of the test. A software can be used to match a user with a proctor, and provide monitoring and quality control over the proctors performance.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, the present application claims priority to U.S. Provisional Patent Application No. 63/363,433, titled “GIG-ECONOMY QUALITY ASSURANCE SYSTEM FOR DATA LABELING AND DISCRETE TASK COMPLETION” and filed on Apr. 22, 2022, U.S. Provisional Patent Application No. 63/336,621, titled “SYSTEMS AND METHODS FOR FACILITATING REMOTE TESTING USING COMPUTER VISION” and filed on Apr. 29, 2022, U.S. Provisional Patent Application No. 63/364,671 titled “SYSTEMS AND METHODS FOR FACILITATING REMOTE TESTING USING COMPUTER VISION” and filed on May 13, 2022, U.S. Provisional Patent Application No. 63/362,576, filed Apr. 6, 2022, and U.S. Provisional Patent Application No. 63/363,358, filed Apr. 21, 2022, each of which is incorporated herein by reference in its entirety for all purposes and forms a part of this specification.

### BACKGROUND

### Field

The present application is directed to remote medical diagnostic testing and testing platforms. Systems, methods and devices for facilitating remote testing, namely for a medical diagnostic test, for a user with the use of computer vision, and quality assurance for data labeling and task completion are also disclosed.

### Description

Use of telehealth to deliver healthcare services has grown consistently over the last several decades and has experienced very rapid growth in the last several years. Telehealth can include the distribution of health-related services and information via electronic information and telecommunication technologies. Telehealth can allow for long distance patient and health provider contact, care, advice, reminders, education, intervention, monitoring, and remote admissions. Often, telehealth can involve the use of a user or patient's personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

A user may undergo (e.g., self-administer) a medical diagnostic test that the user may select from a medical diagnostic test kit container. The user may administer the medical diagnostic test with the aid of a remote medical diagnostic testing platform. A user device (e.g., a smartphone, table, laptop, etc.), of which the remote medical diagnostic testing platform may be implemented on or accessed by, may scan the medical diagnostic testing kit container or medical diagnostic test using computer vision to recognize the type of test being used. In some embodiments, using computer vision, the system may determine whether a proctor can be used for guidance or is required to guide the user through the user's administration of the medical diagnostic test. Once the system determines whether a proctor can be used for guidance through the administration of the medical diagnostic test, the system may direct the user to the appropriate platform accordingly. In cases where a proctor is not necessary, the system may provide the user with other forms of instruction and guidance, such as written, video, or augmented reality guidance, among others.

In some embodiments, computer vision can be used to recognize the diagnostic test being used. Computer vision can recognize the test based on one or more features associate with the test, such as the container, labelling, contents, or machine-readable codes and direct the user to the accordingly. For example, a machine-readable code may include code that identifies the medical diagnostic test or medical diagnostic test kit container as a medical diagnostic test.

In another aspect of the present disclosure, systems, methods, and devices are described that may be provided to ensure quality for data labeling and discrete task completion. As described herein, ensuring quality may include measuring test accuracy and comparing a test accuracy of a worker to a plurality of other workers, or comparing the test accuracy of the worker to a predetermined accuracy threshold.

In some embodiments, the systems, methods, and devices may provide security features such as eye tracking or frame capturing to ensure the accuracy of data labeling, and to ensure quality task completion.

In some embodiments systems and methods for proctor management are described. The systems and methods can be designed to reduce waiting times for users when taking a proctored diagnostic test. The systems and methods can also reduce proctor downtime ensuring that as many proctors as possible are proctoring a medical diagnostic test at one time. The methods and systems may increase the efficiency of proctored diagnostic testing so users can complete a medical diagnostic test in less time, and less proctors are necessary to complete proctored medical diagnostic tests.

In some embodiments, a computer-implemented system for proctor management for a proctored examination platform may include: an electronic storage medium of a computing system, the electronic storage medium may include computer-executable instructions; one or more processors of the computing system, the one or more processors in electronic communication with the electronic storage medium, the one or more processors in electronic communication through an electronic network with a plurality of proctor devices and a user computing device, the one or more processors may be configured to execute the computer-executable instructions stored in the electronic storage medium for implementing the proctor management for the proctored examination platform by: generating, by the computing system, a plurality of routing profiles containing one or more queues, the routing profiles associated with a set of one or more skills of a plurality of proctors and the one or more queues associated with skills of the set of one or more skills of the plurality of proctors, wherein the set of one or more skills of the plurality of proctors includes at least a step of a diagnostic test; receiving, by the computing system, a request from a user computing device of a user for a proctored examination and a set of requested skills; automatically searching, by the computing system, for a plurality of queues including the set of requested skills; placing, by the computing system, the user at an end of an optimal queue, wherein the computing system automatically and dynamically determines the optimal queue based on at least one of, a queue with a shortest wait time, a queue with a most amount of available proctors, or a queue associated with a set of skills most similar to the set of requested skills; connecting, by the computer system, the user device to a video conference session for proctored examination of the diagnostic test including one or more steps when the user if at a front of the optimal queue; and connecting, by the computer system, a first proctor device of a first available proctor associated with a routing profile containing the optimal queue to the video conference session, wherein the user performs each of the one or more steps of the diagnostic test, and the computing system automatically connects at least a second proctor device of a second available proctor with a skill set including at least a current step of the diagnostic test, and wherein the computing system automatically and dynamically disconnects the first available proctor when the second available proctor joins the video conference session.

In some embodiments, a computer-implemented method for matching users with proctors for a proctored examination platform for medical diagnostic tests can include: generating, by the computer system, one or more proctor groups, wherein each of the one or more proctor groups includes one or more proctors, and wherein each of the one or more proctors of a proctor group includes a set of skills, wherein each set of skills includes one or more skills; generating, by the computer system, a routing profile for each of the one or more proctor groups; generating, by the computer system, a queue for each of the one or more proctor groups; assigning, by the computer system, the queue for each of the one or more proctor groups to the routing profile of each proctor group; assigning, by the computer system, to the routing profile of each of the one or more proctor groups, the queues of other proctor groups, wherein the set of skills of each of the one or more proctor groups include each of the one or more skills of the set of skills of the other proctor groups; receiving, by a computer system, a request from a user for a proctored examination of a medical diagnostic test, wherein the request includes a requested set of skills including one or more requested skills; if a proctor group of the one or more proctor groups includes a set of skills, wherein the set of skills is a same set of skills as the requested set of skills, assigning, by the computer system, the user to the queue of the proctor group; if no proctor group of the one or more proctor groups includes a set of skills, wherein the set of skills is the same set of skills as the requested set of skills, assigning, by the computer system, the user to a queue of a proctor group with a highest priority, wherein the set of skills of the proctor group includes every requested skill of the requested set of skills; establishing, by the computer system, a video conferencing session between a user device of the user and a proctor device of a proctor, wherein the proctor is an available proctor of any of the proctor groups including a routing profile that includes the queue of the user.

In some embodiments, the one or more skills of each set of skills can include a language, a step of the medical diagnostic test, a location, and/or a test type.

In some embodiments, the proctor group with the highest priority can be determined by the computer system based on a priority selection of the user.

In some embodiments, the queues of each routing profile can be ranked based on a priority level of the queues.

In some embodiments, the queue with a top priority level can be the queue of the proctor group that includes the routing profile.

In some embodiments, the priority level of the queues can be based on a priority level of one or more of the skills of the set of skills of the proctor group of the queue.

In some embodiments, the priority level of the queues can be based on a similarity of the set of the skills of the proctor group of that includes the routing profile and the set of skills of the proctor group of the queue.

In some embodiments, the proctor can be a first proctor, wherein the first proctor observes, via the video conferencing session, the user performing a first step of the medical diagnostic test.

In some embodiments, the computer-implemented method can further include the step of after the first step of the medical diagnostic test is completed by the user, connecting, by the computer system, a second proctor to the video conferencing session, to observe the user performing the second step of the medical diagnostic test.

In some embodiments, the computer system can be configured to display a computer-generated representation of a proctor to the user via the user device, and wherein the computer generated representation of the proctor is a same computer generated representation for the first proctor and the second proctor.

In some embodiments, a computer-implemented method for matching users with proctor for a proctored examination platform for medical diagnostic tests can include: generating, by the computer system, a proctor group, wherein the proctor group includes one or more proctors, and wherein each of the one or more proctors of the proctor group includes a set of skills, wherein the set of skills includes one or more skills; generating, by the computer system, a routing profile for the proctor group; generating, by the computer system, a queue for each of the one or more proctor groups; assigning, by the computer system, the queue to the routing profile; assigning, by the computer system, to the routing profile, queues of other proctor groups, wherein the set of skills of the proctor group includes every skill of a set of skills of the other proctor groups; ranking, by the computer system, each queue of the routing profile based on a priority level of each queue, wherein a queue with a top priority level is the queue of the proctor group, and wherein a priority level the queues of the other proctor groups is based on a number of skills of the set of skills of the proctor group of the routing profile not in the set of skills of the other proctor groups; establishing, by the computer system, a video conferencing session between an available proctor of the proctor group and a highest priority user, wherein the highest priority user includes a user in a queue with a highest priority.

In some embodiments, the one or more skills of the set of skills of the proctor group can include a language, a step of the medical diagnostic test, a location, and/or a test type.

In some embodiments, the queue with the highest priority can include a non-empty queue with the highest priority level.

In some embodiments, a computer-implemented system for matching users with proctors for a proctored examination platform for medical diagnostic tests can include: at least one processor in communication with at least one memory storing instructions that cause the at least one processor to implement the proctored examination platform by: generating, by the computer-implemented system, one or more proctor groups, wherein each of the one or more proctor groups includes one or more proctors, and wherein each of the one or more proctors of a proctor group includes a set of skills, wherein each set of skills includes one or more skills; generating, by the computer system, a routing profile for each of the one or more proctor groups; generating, by the computer-implemented system, a queue for each of the one or more proctor groups; assigning, by the computer-implemented system, the queue for each of the one or more proctor groups to the routing profile of each proctor group; assigning, by the computer-implemented system, to the routing profile of each of the one or more proctor groups, the queues of other proctor groups, wherein the set of skills of each of the one or more proctor groups include each of the one or more skills of the set of skills of the other proctor groups; receiving, by the computer-implemented system, a request from a user for a proctored examination of a medical diagnostic test, wherein the request includes a requested set of skills including one or more requested skills; if a proctor group of the one or more proctor groups includes a set of skills, wherein the set of skills is a same set of skills as the requested set of skills, assigning, by the computer-implemented system, the user to the queue of the proctor group; if no proctor group of the one or more proctor groups includes a set of skills, wherein the set of skills is the same set of skills as the requested set of skills, assigning, by the computer-implemented system, the user to a queue of a proctor group with a highest priority, wherein the set of skills of the proctor group includes every requested skill of the requested set of skills; establishing, by the computer-implemented system, a video conferencing session between a user device of the user and a proctor device of a proctor, wherein the proctor is an available proctor of any of the proctor groups including a routing profile that includes the queue of the user.

In some embodiments, the one or more skills of each set of skills can include a language, a step of the medical diagnostic test, a location, and/or a test type.

In some embodiments, the proctor group with the highest priority can be determined by the computer system based on a priority selection of the user.

In some embodiments, the queues of each routing profile can be ranked based on a priority level of the queues.

In some embodiments, the queue with a top priority level can be the queue of the proctor group that includes the routing profile.

In some embodiments, the priority level of the queues can be based on a priority level of one or more of the skills of the set of skills of the proctor group of the queue.

In some embodiments, the priority level of the queues can be based on a similarity of the set of the skills of the proctor group of that includes the routing profile and the set of skills of the proctor group of the queue.

In some embodiments, the computer-implemented system can include at least one processor and at least one non-transitory storage medium storing instructions that are executed by the processor.

For purposes of this summary, certain aspects, advantages, and novel features of the invention are described herein. It is to be understood that not necessarily all such advantages may be achieved in accordance with the particular embodiments of the invention. Thus, for example, those skilled in the art will recognize that the invention may be embodied or carried out in a manner that achieves one advantage or group of advantages as taught herein without necessarily achieving other advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular disclosed embodiment(s).

### BRIEF DESCRIPTION OF THE DRAWINGS

These and other features, aspects, and advantages of the present application are described with reference to drawings of certain embodiments, which are intended to illustrate, but not limit the present disclosure. It is to be understood that the attached drawings are for the purpose of illustrating concepts disclosed in the present application and may not be to scale.

FIG. 1 illustrates an example of an at-home medical diagnostic test kit container or package having a machine-readable code that can be scanned to initiate the administration of a test according to some embodiments described herein.

FIG. 2 illustrates an example of a user scanning the machine-readable code on the medical diagnostic test kit container of FIG. 1 using a camera of a user device (such as a mobile phone), as well as an example graphical user interface shown on a display of the user device after the machine-readable code has been scanned, according to some embodiments described herein.

FIG. 3 illustrates an example of a user scanning a machine-readable code located on a particular medical diagnostic test kit using a mobile phone having a built-in camera according to some embodiments described herein.

FIG. 4 is a block diagram illustrating a process for a system using computer vision to determine whether a proctoring platform is required for administration of a test and direct the user accordingly.

FIGS. 5A-5C represent user displays during an embodiment of an image-based test kit recognition process.

FIG. 6 is a block diagram illustrating an embodiment of a computer hardware system configured to run software for implementing one or more embodiments of the health testing and diagnostic systems, methods, and devices disclosed herein.

FIG. 7 is a block diagram that illustrates an example process for determining qualifications of a prospective worker.

FIG. 8 is a block diagram that illustrates an example process for determining qualifications of an existing worker.

FIG. 9 is a block diagram that illustrates an example process for evaluating existing workers or prospective workers.

FIG. 10 illustrates a schematic of an example management system according to some embodiments described herein.

FIG. 11A illustrates a schematic of an example proctor queuing system according to some embodiments described herein.

FIG. 11B illustrates a schematic of another example proctor queuing system according to some embodiments described herein.

FIG. 11C illustrates an example schematic of a proctor according to some embodiments described herein.

FIG. 12A illustrates an example flowchart of a method for adding a proctor to a proctor group according to some embodiments described herein.

FIG. 12B illustrates an example flowchart of a method for matching proctors to users in a medical diagnostic platform according to some embodiments described herein.

### DETAILED DESCRIPTION

Although several embodiments, examples and illustrations are disclosed below, it will be understood by those of ordinary skill in the art that inventions described herein extend beyond the specifically disclosed embodiments, examples, and illustrations and includes other uses of inventions obvious modifications and equivalents thereof. Embodiments of the inventions are described with reference to accompanying figures, wherein like numerals refer to the like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

As mentioned briefly above and as will now be explained in more detail below with reference to the example embodiments provided in the figures, this application describes systems, methods, and devices that are configured to facilitate remote testing for a user with the use of computer vision, such as medical diagnostic testing. Embodiments of the inventions described herein can comprise several novel features and no single feature is solely responsible for the desirable attributes or is essential to practicing the inventions described.

Remote testing, such as remote medical diagnostic testing, can be used upon the user selecting a test to administer. In some instances, the test selected may be within a medical diagnostic test kit container or package that functions similarly to a first aid kit but includes several at-home medical diagnostic test kits of different types that a household (e.g., an individual or family), care facility or assisted living center, or other group or individual, could use to complete at-home medical testing. The diagnostic test kit container or package may be purchased from an e-commerce provider or a retail store without requiring a prescription and stored and used on demand as needs arise for diagnostic or medical testing. For example, if an individual is feeling sick or experiencing symptoms of a medical condition (e.g., COVID-19, influenza, urinary tract infection, etc.), the individual may open the medical diagnostic test kit container and select an appropriate at-home diagnostic test and complete the at-home medical diagnostic test. The at-home medical diagnostic tests may be self-administered, administered by another individual in the household or care facility, or a health care provider.

In some instances, the medical diagnostic test kits may also be used even in instances where no symptoms are being experienced, as desired. In addition, if a negative screening test is required prior to travel or employment (e.g., such as a negative COVID-19 test, a negative drug test, etc.), the medical diagnostic test kit container may include screening tests that are certified and accepted as proof by government agencies, travel companies and institutions (e.g., airlines, cruise ship companies, etc.), restaurants, gyms, fitness centers, hospitals, other retail establishments, and employers that may require proof of negative diagnostic tests.

In some instances, different medical diagnostic test kit containers or packages may include various types and numbers of tests designed for different individuals, households, car facilities, etc. A label on the diagnostic test kit container may indicate the types and numbers of medical diagnostic tests in the particular container or package. In addition, supplemental medical diagnostic test kits may be ordered (e.g., via an e-commerce website or platform) when needed to restock supply. Certain medical diagnostic test kits may require a prescription or approval from a health care provider prior to ordering. Other medical diagnostic test kits may be purchased without any prescription or approval at the time of purchase.

The medical diagnostic test kit container or package may include a machine readable code located on an external surface of the box that can be scanned by a camera of a user device (such as a personal computer, a cellular phone, a smartphone, a laptop, a tablet computer, smart glasses, an e-reader device, an audio player, or another device capable of connecting to and communicating over a network, whether wired or wireless) to initiate remote testing with the use of computer vision to determine whether a proctor may be used for the administration of the medical diagnostic test.

In some instances, for example, if a proctor may be used for the administration of the medical diagnostic test, a remote health testing and diagnostic platform (e.g., a digital point of care platform operated by a commercial entity, such as eMed based in Florida) may also be used to facilitate coordination with proctors to enable certification of certain diagnostic tests, to facilitate ordering and delivery of prescription medicine that may be helpful to treat a medical condition identified by a medical diagnostic test in the medical diagnostic test kit package, to facilitate generation of a certification or pass that can be displayed as proof of a negative screening test (such as a negative COVID-19 test), to facilitate re-ordering or supplemental ordering of medical diagnostic test kits when inventor of a particular medical diagnostic test kit type in the medical diagnostic test kit container or package is low or out of stock, and/or to generate pre-screening surveys to determine whether or not it makes sense for the user to complete a particular medical diagnostic test.

In some instances, a user may select a medical diagnostic test to administer from the medical diagnostic test kit container or package. For example, the user may select a COVID-19 diagnostic test, influenza diagnostic test, urinary tract infection diagnostic test, or other medical diagnostic test. In some instances, the user may use a camera on a user device to scan the medical diagnostic test selected. This may allow a system to identify the medical diagnostic test that the user selected. The system may use computer vision to identify the medical diagnostic test selected by the user. As described above, the medical diagnostic test may include machine readable code that may include computer recognition information on the outside of the medical diagnostic test. This may allow the system to use computer vision to identify the medical diagnostic test chosen by the user. Such computer recognition information may include the box of which the medical diagnostic test is held within, a QR code, AR code, bar code, data matrix code, PDF417 code, Aztec code, materials contained within the medical diagnostic test box, a lot number provided by the Food and Drug Administration (“FDA”), etc. The identification of the selected medical diagnostic test may allow the system to register the medical diagnostic test selected. For example, registration of the medical diagnostic test selected can allow the system to provide the user with a corresponding administration of the medical diagnostic test that may be tailored to the medical diagnostic test selected by the user. In some instances, the system may determine whether a proctor may be used for administration of the medical diagnostic test. Depending on the medical diagnostic test chosen by the user, a proctor may be used to guide the user in administration of the medical diagnostic test. For example, if the system determines a proctor may be used to guide the user in administration of the medical diagnostic test, the system may direct the user to a proctoring platform, as described above. In some instances, if the system determines that a proctor may not be used to guide the user in the administration of the medical diagnostic test chosen, the system may direct the user to other forms of guidance. Such guidance can include instructions for self-guided administration, or other instructions.

In some instances, the system may guide or instruct the user as to which medical diagnostic test to select for administration. This may include the user informing the system which symptoms the user may be experiencing. With this information, the system may determine which medical diagnostic test may be appropriate for the user to administer for diagnostic purposes. Easing the process of the administration of a medical diagnostic test may be important for reducing administration errors and ensuring accurate medical diagnostic test results. Accordingly, it may be beneficial to provide the user with a system that utilizes computer vision to direct the user to the proper platform to administer a medical diagnostic test.

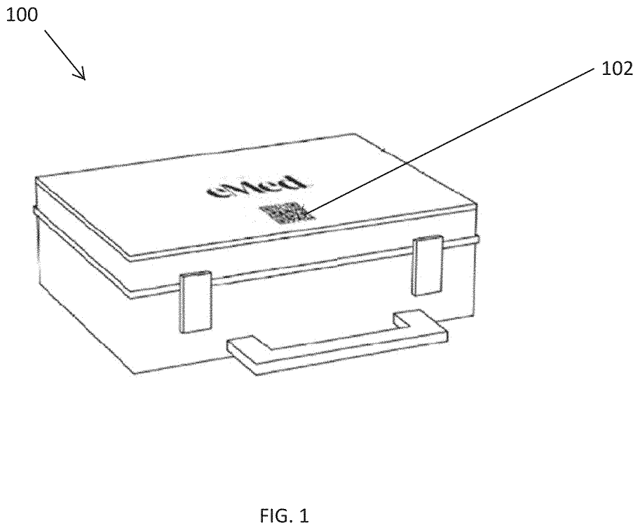

FIG. 1 illustrates an example of an at-home medical diagnostic test kit container or package 100 that may be utilized in conjunction with a remote health testing and diagnostic platform. The medical diagnostic test kit container or package 100 may function similar to a first aid kit but, instead of including bandages, gauze pads, wraps, ointments, or other typical first aid kit supplies, the medical diagnostic test kit container or package 100 includes a plurality of medical diagnostic test kits of various types that can be used to perform at-home or self-administered medical diagnostic tests. The diagnostic test kit container 100 may comprise a box, package, canister, or other container adapted to hold and store contents. The diagnostic test kit container 100 may be made of plastic, cardboard, metal, polymeric, or other material.

The medical diagnostic test kit container 100 may include one or more graphics that may be computer recognition information (e.g., images and/or alphanumeric text). The graphics may be printed on an exterior surface of the medical diagnostic test kit container or may be printed on an adhesive label or sticker that is adhered to the exterior surface of the medical diagnostic test kit container. The graphics may include, among other things, a machine-readable code 102 (e.g., QR code, AR code, bar code, data matrix code, PDF417 code, Aztec code) that, when scanned or imaged by a user device, such as a mobile computer device having one or more built-in cameras, causes the user device to provide information about the collection of medical diagnostic test kits within the medical diagnostic test kit container 100 as a whole, about the individual medical diagnostic test kits, and/or about the medical diagnostic tests themselves. In some embodiments, the machine-readable code 102 may correspond to a graphic (e.g., an image, logo, etc.) that an application or web app may recognize using one or more computer vision techniques to provide one or more pieces of the aforementioned information. The machine-readable code 102 may be located at any area of the medical diagnostic test kit container 100.

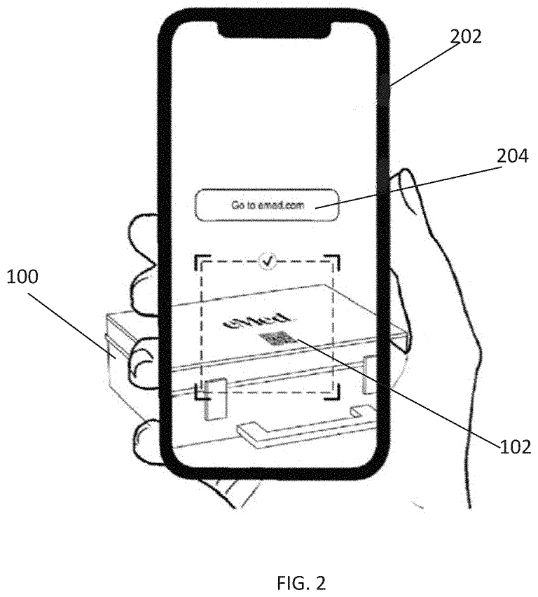

FIG. 2 illustrates an example of an individual scanning the machine-readable code 102 on the medical diagnostic test kit container 100 using a built-in camera (e.g., rear-facing camera) of a user device 202 (such as a mobile phone, smartphone, tablet, laptop, e-Reader device, smartwatch, smart glasses). The real-time video images obtained by the camera(s) of the user device can be displayed on a display screen of the user device. This may allow the system to utilize computer vision to determine whether a proctor may be used for the medical diagnostic test selected by the user from the medical diagnostic test kit container 100.

In some embodiments, a graphical user interface 204 may be shown on a display of the user device after the machine-readable code has been scanned, which allows the user to navigate to a website or mobile application containing information about proctoring or self-administration of the medical diagnostic test selected by the user from the medical diagnostic test kit container 100.

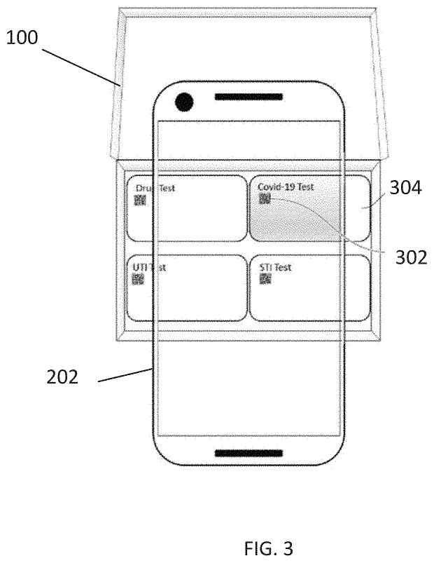

FIG. 3 illustrates an example of a user scanning a machine-readable code 302 located on a particular medical diagnostic test kit 304 (in this instance, a COVID-19 test kit) using computer vision with a user device 202 (e.g., mobile phone) having one or more built-in cameras. The machine-readable codes 302 may be scanned while a particular medical diagnostic test kit 304 remains within the medical diagnostic test kit container 100 or after removal of the particular medical diagnostic test kit 304.

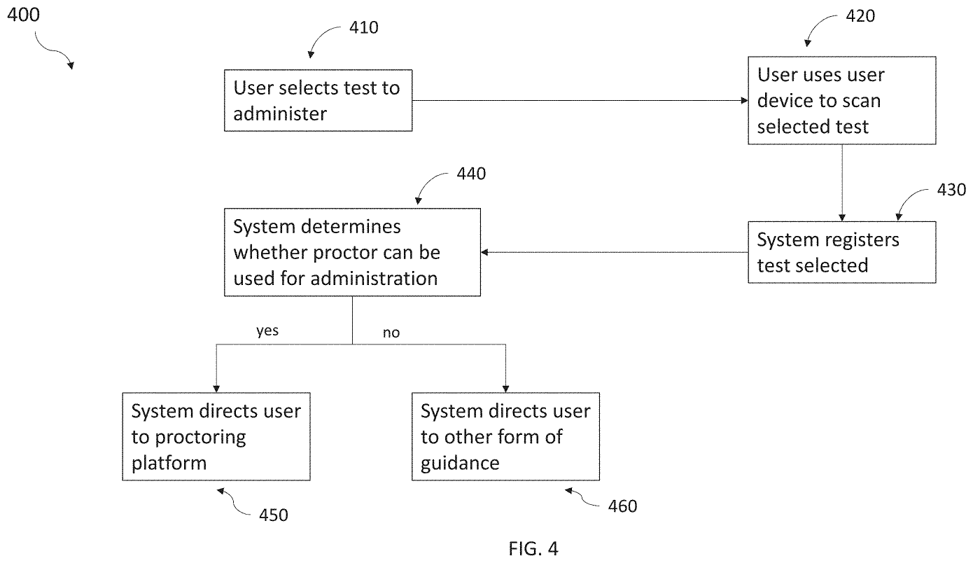

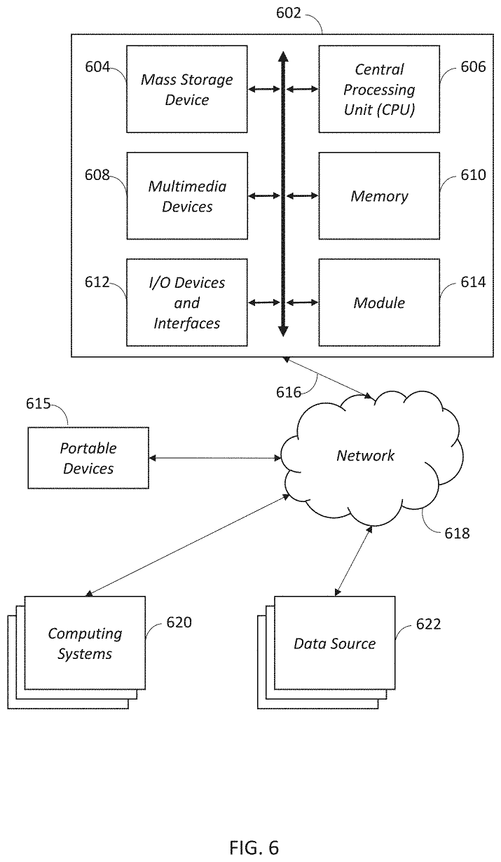

FIG. 4 illustrates a block diagram of an example of a protocol or method 400 for a system using computer vision to determine whether a proctoring platform is required for administration of a test and direct the user accordingly. The method 400 can be implemented, for example, using one or more components of the system shown in FIG. 6.

At block 410, a user may select a medical diagnostic test from the medical diagnostic test kit container to administer. As described above, the medical diagnostic test and/or medical diagnostic test kit container may include machine-readable code, which may allow for the use of computer vision. At block 420, the user may use a user device (e.g., mobile phone) to scan the selected test. The user device may include one or more built-in cameras, which may implement computer vision to identify and interpret the machine-readable code.

At block 430, through the initial implementation of computer vision, a system may register the medical diagnostic test selected by the user from the real-time video images obtained by the user device. At block 440, the system may determine whether a proctor can be used for guidance during the administration of the medical diagnostic test. This may depend on the medical diagnostic test selected by the user. Using the initial implementation of computer vision, the system may be able to determine whether the medical diagnostic test selected by the user can use a proctor for guidance during the administration of the medical diagnostic test. At block 450, the system may determine a proctor can be used for guidance during the administration of the medical diagnostic test selected by the user and direct the user to a proctoring platform. The proctoring platform can provide a qualified proctor to guide the user through the particular administration of the medical diagnostic test selected by the user.

At block 460, the system may determine a proctor may not be used for guidance during the administration of the medical diagnostic test selected by the user. The system may then direct the user to a platform that may provide other forms of guidance to the user, such as self-guided instructions.

Although several of the examples provided above have described the user scanning a machine-readable code on the test kit to enable the system to determine the type of test being administered, in other examples, computer vision may be used to analyze other features of a test kit to determine its type. For example, computer vision can be used to analyze the shape and size of the packaging, contents of the test kits, labels included on the test kit, etc., to determine its type.

In some embodiments, a user may capture an image, images, or video of a box or package of a test kit. The image, images, or video can be analyzed to determine which type of test kit the user is using. For example, when the user is ready to scan a test box, the user's device can load a website that creates a real-time communication connection to a service running on a computer system (e.g., a cloud-based computer). The computer system may also be connected to a separate website (which could have password protection or other access limitations) where photos (e.g., of new boxes) can be uploaded, turned into templates, and labeled/tagged with other information (e.g., whether each test is supported by the testing service). Such data can thus be made available to the computer system. Alternatively, or additionally, templates may be separately generated using known box photos, labeled, and uploaded into a system library which is accessible by the computer.

In some embodiments, when the video connection starts, the user device camera is turned on and sends user video/image data to the computer system. The computer system then receives user video/image data (i.e., a series of user images) frame by frame. The computer system can select one or more of the user images for processing. In some embodiments, the computer system may select every image received from the user device or may select a subset of the images received (e.g., every sixth image). In some embodiments, unused images may be discarded or stored for possible use in subsequent comparison processes.

Next, in some embodiments, the selected user images are compared to one or more templates. Such comparison can be made by the computer system. For example, a first selected user image is sent to the computer system for comparison with multiple templates (e.g., templates A-D based on known test boxes and stored in a template library). The computer system may simultaneously compare the first selected image with template A (e.g., at process A), template B (e.g., at process B), template C (e.g., at process C), and template D (e.g., at process D) using parallel processing. More or fewer simultaneous processes can be enabled depending on the particular capabilities of the computer system (e.g., the type of processor used by the computer system). The multiple processes A-D can also be run sequentially (in batches or individually) if there are more templates than CPU cores.

In some embodiments, no image pre-processing is done, but a step could be added to detect the presence of a box before sending the image to the comparison algorithm, which may be a scale-invariant feature transform (SIFT) algorithm. For example, for each of the comparison processes, a SIFT algorithm is performed to identify features of the selected user image and features of the template images.

The selected user image and the template image can remain in a color saturated state or can be desaturated before features are identified. In some embodiments, both the color and desaturated images are used to create a template and/or a color profile (as discussed with respect to a second process below).

In some embodiments, next a Fast Library for Approximate Nearest Neighbors (FLANN) algorithm can receive the results of the SIFT algorithm and quantify how close the features of the selected user image match features in the templates. The result of the FLANN algorithm is a number less than or equal to 1 and numbers closer to 1 represent a better match between the selected image features and the template features.

In some embodiments, the order of comparisons of the templates can be automatically (or manually) prioritized such that most common template matches are tested first. This can be accomplished by placing higher occurring tests closer to the beginning of the array of templates.

The number of possible feature matches depends on the particular box design. For example, the acceptable number of feature matches between a selected user image and the template for a very simple box could be around seventy while the acceptable number of feature matches for a more complicated box design (e.g., with more printed shapes, lines, text, colors, etc.) could be around one hundred and forty. These numbers of matches are provided only by way of example and other numbers much lower or much higher could be used.

In some embodiments, if the acceptable number of feature matches is low (e.g., for the simpler box design template), the FLANN algorithm can be performed multiple times to confirm results. For box templates with a higher number of features and thus higher feature match requirements, fewer passes through the algorithm may be required before the result is considered reliable. In general, the templates/boxes that have the highest number of accepted matches after the SIFT to FLANN analysis can be prioritized as long as the FLANN-based match count is above the required threshold for that particular box/template.

In some embodiments, a threshold for the FLANN output number can be set to define what level of feature matching is acceptable. For example, a FLANN result greater than 0.8 or 0.9 can be considered a reliable match between the two images. Other thresholds may also be used.

If there are multiple results from processes A-D that meet respective set thresholds A-D, comparison processes associated with the positive matches can be performed again (using a new user image) to confirm results. If the user input is a video feed, the system has enough unique frames to pull from without requiring any additional user input; if the user input is a still image, the user may be prompted to provide another image.

If there are multiple tests that meet the threshold, the test with the most matches after FLANN can be prioritized as the most likely box/template match, assuming the difference between numbers of feature matches is significant (e.g., seventy matches to one template vs one hundred and forty matches to another template). If there are still multiple results that meet thresholds, the comparison process above is unable to identify a single most likely box pictured within the selected image. Another possible outcome is that the user image does not meet the threshold for any of the known box images.

If the system cannot determine a single template match, one or more of the following steps can be performed: (a) generation and display of a notification instructing the user to take a new photo/video of their test box under different lighting conditions, nearer to the box, with a clean camera lens, at a different angle, etc., then running the comparison process again with better user images; (b) if there are multiple possible positive matches, select whichever result has the highest number of feature matches; (c) use a second comparison process based on different characteristics of the user image/template or different pre-processed versions of the images.

The second comparison process could be performed if multiple runs of the first comparison process generate different results (e.g., first result meets threshold, second result does not meet threshold, third result meets threshold, etc.). The second comparison process could be used as a double check on the results of the first comparison process. The second comparison process may be based on color. For example, each known box image can have an associated color profile (e.g., 85% of the front of the box is yellow, 15% of the front of the box is blue) and a color profile can be generated for the user input image.

In some embodiments, edges/bounds of the test box in the user device image can be determined using the SIFT algorithm and then a user image color profile based on the saturated pixels within the identified box bounds is created. If the user image color profile matches the known box color profile within a set tolerance, a box match condition can be confirmed.

The system can ask the user to directly select which test box they have. When asking the user to select a box, the user may be presented with all known box images to choose from or may be presented with only the known box images that were not eliminated by the comparison processes for failing to meet threshold requirements.

In some embodiments, once the system has determined which test box the user has, an image of the test box can be displayed to the user. In some embodiments, the user can provide input to confirm the result. The identified test box can have an associated name/brand or other tags, such as whether or not the test is supported by the proctored testing service. If the test is a supported test, the user is presented with the option to begin a testing experience. If the test is not a supported test, the user is presented with the option to purchase a supported test.

In some embodiments, the color comparison process could be used before the feature comparison process in some embodiments, though it may be preferable to do the feature detection and matching first. The above description assumes that the user is taking a picture of a single test box. Variations are possible where two or more tests are present in the user's image. The multiple distinct boxes can be recognized and compared to templates individually using the comparison processes described. Results displayed to the user could include information about which tests were identified, and which of the identified tests are supported by the testing service. This embodiment could be useful if a user is in a store or pharmacy and is looking at multiple brands/types of COVID tests on the shelf. The user could scan multiple tests to identify which brands are supported, and this can help a user make a purchasing decision.

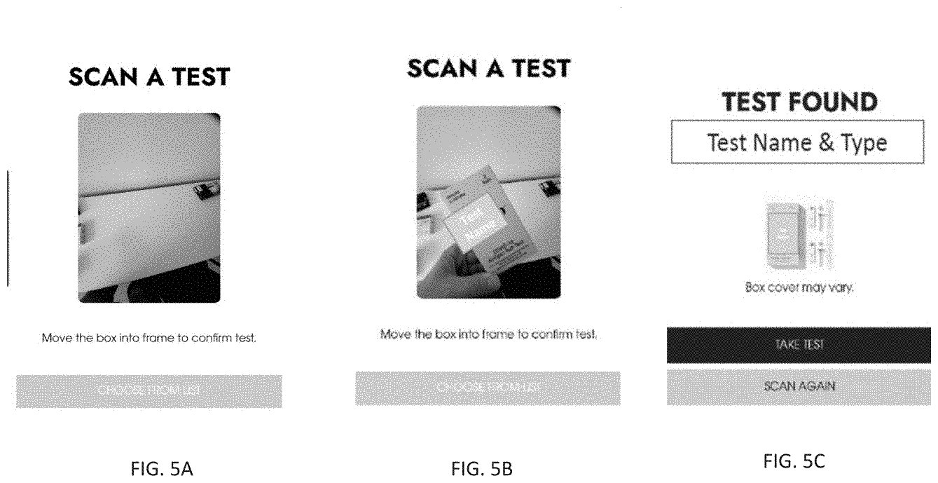

FIGS. 5A-5C illustrate example displays provided on a display of a user device during the method described above. In FIG. 5A, the user is instructed to capture an image of the test box. In FIG. 5B, the user moves the test box into the frame and the image is captured. The image is then analyzed as described above and a match is determined. In FIG. 5C, the match is presented to the user for confirmation.

### Quality Assurance for Data Labeling and Discrete Task Completion

At times, workers can be needed on a short-term basis, during irregular hours, or otherwise under conditions in which it may be desirable to make use of non-traditional workers. For example, “gig” workers often perform tasks such as transportation, food delivery, and so forth. Often, these tasks are fairly simple, for example driving from a first location to a second location or driving to a restaurant or grocery store to pick up a delivery and then driving to a customer's home or place of work to complete the delivery.

Gig workers can often receive little training and operate without much (or any) direct supervision. For some tasks, such an approach can work well. For example, once a delivery driver is used to operating the user interface of an application through which the delivery driver receives instructions to pick up and deliver orders, there is little need for additional training or monitoring. In some cases, customers can act as a check on the gig worker's performance. For example, a customer of a ride sharing service may leave a review or rating indicating the customer's satisfaction with the gig worker's performance. In some cases, the customer can provide specific feedback, such as noting that the gig worker's car was clean or dirty, that the gig worker drove carefully or carelessly, that the driver did not appear to be the person in a photo shown to the customer in the application associated with the service, and so forth. As another example, a food delivery driver can receive ratings that indicate a customer's satisfaction with the delivery experience. For example, a customer may rate a driver based on the delivery experience. In some cases, a customer can leave feedback, such as identifying a late or early delivery, identifying issues such as missing items, spilled containers, drop off at the wrong address, and so forth.

In the scenarios described above and in other similar scenarios, limitations on training and supervision of gig workers can be less impactful because the tasks being completely are relatively simple and customers can provide feedback that can be used to identify any problematic gig workers who should undergo further training or be removed from a service that uses gig workers (for example, a ride share driver with a consistently low rating can be disallowed from working with the ride share service again, either permanently or for a limited period).

In some circumstances, it can be desirable to use gig workers (or similar short term, irregular, or otherwise non-standard employment) to carry out more complex tasks. For example, during a health emergency, there can be a significant demand for individuals who can help perform diagnostic testing. In some cases, gig workers can be used for events where more complex tasks, such as checking credentials (e.g., identification, admittance tickets, and so forth) to verify that an individual is who they say they are and that the individual has permission to enter a venue or event.

While customers can provide feedback that can be used to monitor gig worker performance in scenarios such as ride share services and delivery services, this may not be a viable solution in other circumstances. For example, an individual who has a diagnostic test performed by a gig worker may lack the expertise to know if the gig worker is doing a good job or it. For example, the individual may not be aware of the proper procedure for administering a test or how to interpret the test result. For example, in the case of a test that uses a nasal swab for sample collection, the individual may not know how far the swab should be inserted, whether or not the swab should be rotated while in the nostril, whether or not both nostrils should be swabbed, and so forth. In some cases, the individual may not know how to interpret a test result. For example, an individual may not be aware of how many lines should be present on a lateral flow test strip to determine whether a result is positive, negative, or invalid. In many cases, the individual may not even see the test strip and may instead receive only a positive, negative, or invalid result, with no way to verify whether the worker interpreted the test result correctly. Similarly, in the case if identification or other credential verification, an individual may not notice if a worker actually verified their identification or credential, if the worker performed all the checks that they were supposed to carry out, and so forth. Similarly, an individual who seeks to enter a venue, event, or the like without authorization is unlikely to report that a worker failed to check their identification, ticket, or the like.

Thus, in some circumstances, feedback from individuals who have interactions with a gig worker may not be a viable means of monitoring the performance of the gig worker. Accordingly, there is a need for systems, methods, and devices that can be used to determine which tasks should be assigned to particular gig workers, to determine if a gig worker is performing their job adequately, to determine if the gig worker is actually performing the assigned tasks (as opposed to, for example, getting a friend, relative, or other individual to complete tasks for them), and so forth.

In the context of telehealth, a proctor may have a variety of tasks to complete. For example, at the beginning of a telehealth session, the proctor can verify the identity of a patient, for example by comparing the person appearing in a video feed to an image of a license or other identification document. In some embodiments, the patient can present the identification document via the video feed, for example by holding up the patient's license so that it is within the field of view of a camera of a user device. In some embodiments, the patient can take an image or scan the identification document, which can be provided to the proctor. For example, the patient can capture an image of the identification document prior to the testing session (and, for example, upload the image to a telehealth portal where it can be associated with a patient profile of the patient). In some embodiments, the patient may capture an image of the identification document during the telehealth session.

The proctor can verify various information on the identification document. For example, the proctor can verify that the patient and the photograph appearing on the identification document appear to be the same person. The proctor can also verify other information, such as the patient's date of birth, address, height, weight, and so forth. For example, the proctor can compare the information against information in a patient profile. The patient profile information can originate from a variety of sources. For example, any combination of the patient, the patient's employment, an event organizer, and so forth can provide information to the telehealth platform which can become part of the patient's profile.

In some embodiments, the proctor may observe part or all of a testing procedure. For example, the proctor can observe a sample collection procedure, a sample analysis procedure, and so forth. For example, in the case of a nasal swab collection procedure, the proctor can observe the sample collection to ensure that the patient inserts the swab to the correct depth, rotates the swab a certain number of times, swabs both nostrils, and so forth. In the case of a mouth swab, the proctor can ensure that the patient swabs a suitable area of the mouth for a suitable amount of time, for example. Similarly, a proctor may ensure that a patient performs a throat swab procedure correctly. These are merely examples, and it will be appreciated that monitoring may be desirable or even necessary (for example, sample collection monitoring can be required for some types of testing or when a test result is to be used for a particular purpose) for a wide variety of tests which can have a variety of sample collection procedures.

A proctor can also monitor an analysis procedure. For example, after sample collection, a patient can be instructed to place a swab in a solution, apply drops of the solution to a test card or test strip, and so forth. The patient can be instructed to wait a period of time (e.g., 5 minutes, 10 minutes, 15 minutes, and so forth) before reading the results.

In some embodiments, a proctor can interpret results. For example, the patient may place a test card or test strip within a field of view of the camera of the user device. In some embodiments, the proctor can observe the test card, test strip, or the like and can make a determination of whether the test result is positive, negative, or invalid. For example, for a lateral flow test, it is common for one line (a control line) to indicate a valid, negative test, for two lines (a control line and a test line) to indicate a valid positive test, and for no line to indicate an invalid test.

The tasks that can be performed by a proctor can benefit from attention to detail, training, attentiveness, and so forth. For example, for verifying identification, it can be important for the proctor to carefully inspect the identification document to ensure that information in the patient profile is correct, to ensure that the individual is who they say they are, to ensure that the document itself is not fraudulent, and so forth. While some kinds of verification can be relatively straightforward, such as matching a birth date in a patient profile with a birth date shown on the identification document, other kinds of verification may be more difficult or may not have clear answers. For example, a patient's photo on an identification document may differ slightly or markedly from the appearance of the patient during the testing session. The photo on the identification document may be several years old, and the patient's appearance may have changed. For example, the patient may have gained or lost weight, changed hair color, changed hair style, changed facial hair, undergone a cosmetic procedure, started or stopped wearing eyeglasses, changed eyeglass frame style, and so forth.

As described above, there are many tasks that can be performed by remote proctors or other remote workers, gig workers, and so forth. In some cases, workers may perform relatively complex tasks. In some cases, a worker's performance may not be susceptible to the kinds of monitoring typically used for gig work, remote interactions, and the like, such as user ratings and reviews. Moreover, there can be more significant consequences if mistakes or oversights are made. For example, in the case of remote testing for an infectious disease, an incorrect negative test result could result increased spread of the infectious disease as an individual who is positive for the infectious disease may travel, socialize, and so forth, believing that they do not have the infectious disease. As another example, in the case of drug testing, poor monitoring can result in impaired workers remaining on the job, where they may place themselves or others in danger. As another example of drug testing, if employment decisions (e.g., a decision to hire or not hire, or a decision to terminate an existing employee) is based on the result of a drug test, there could be significant liability issues if the employer or testing service cannot establish that the drug test was administered properly.

It can be important to monitor the performance and qualifications of prospective workers and existing workers. For example, in the case of prospective workers, an initial qualification procedure can determine what types of tasks the worker can perform. In the case of existing workers, it can be important to monitor performance to detect, for example, that a worker's performance has decreased, for example because the worker has become bored and is paying less attention. In some embodiments, monitoring existing workers can be used to qualify a worker to perform additional tasks. For example, a worker who has demonstrated high performance in one type of tasks may be assigned other tasks that are similar and/or that have greater complexity. Preferably, tasks can be dynamically assigned to workers based at least in part on the qualifications to perform the tasks, thereby enhancing task performance quality. In some cases, such an approach can lead to improved efficiency as workers are better able to focus on tasks they are good at or enjoy performing.

### Qualification Overview

In some embodiments, qualification can be based on evaluations using questions that have clear correct or incorrect answers. For example, confirming a date of birth in a high quality image of an identification document can have a clear right or wrong answer. In some embodiments, qualification can be based on evaluations using questions that do not have clear correct or incorrect answers. For example, confirming a date on a low quality image of an identification document may not have a clear right or wrong answer. Similarly, confirming a test result in a blurry, fuzzy, or poorly lit image may not have a clear correct or incorrect answer. In some embodiments, prospective workers can be evaluated using a mix of questions that have clear answers and a mix of questions that do not have clear answers.

In some embodiments, questions may be shown to multiple individuals (e.g., multiple prospective workers, multiple other individuals, or a combination of prospective workers and other individuals). In some embodiments, the answers received from the multiple individuals can be used to determine a “correct” answer to a question. For example, a plurality of workers, prospective workers, other individuals, or any combination of individuals can form a consensus pool. In some embodiments, a consensus can be form when criteria are met. Criteria can include, for example and without limitation, a number of individuals who have answered the question, a consensus rate, and so forth. In some embodiments, for example, a question may not be used for qualification purposes until a threshold number of individuals have provided answers to the question. In some embodiments, a question may not be used for qualification purposes until the fraction of individuals answering the question in a particular manner reaches or exceeds a threshold amount. For example, a system can use a question for evaluation purposes after 10, 20, 30, 50, 50, 100, 1000, 10,000, or more individuals, or any number between these numbers, answer a question. For example, a system can use a question for evaluation purposes after 60%, 70%, 80%, 90%, 95%, 99%, or some other percentage of individuals agree on an answer. In some embodiments, both number of individuals answering and percentage of agreeing answers can be used. For example, for a smaller pool of respondents, a system can be configured to require a higher rate of agreement, while for a larger pool of respondents, it may be acceptable to use a question even though the percentage of individuals responding in a particular manner is smaller. In some embodiments, a question can be used for evaluation purposes if a confidence level meets or exceeds a threshold value.

In some embodiments, a plurality of tasks can be separated into a plurality of categories. In some embodiments, an accuracy or performance of a prospective worker can be measured for one or more of the plurality of categories. In some embodiments, the accuracy or performance of the prospective worker can be determined based on, for example, a number of questions or percentage of questions answered correctly by the prospective worker. In some embodiments, the accuracy or performance of the prospective worker can determine a level of compatibility with one or more categories of the plurality of categories. In some embodiments, the level of compatibility can be used to determine which of plurality of tasks the prospective worker can be assigned to perform. In some embodiments, if a worker has a higher level of compatibility with one of the plurality of categories, the worker can be assigned to tasks in said category preferentially. For example, the worker can be assigned tasks in said category whenever such tasks are available and the worker is available to complete tasks. In some embodiments, said category can make up a substantial portion (for example, half or all) of the worker's assigned tasks. In some embodiments, other tasks can be assigned to the worker when there are insufficient tasks available in said category. In some embodiments, the worker may be assigned tasks in other categories in order to help prevent the worker from becoming bored, ceasing to pay adequate attention when performing tasks, ceasing to perform tasks due to burn out, and so forth.

### Prospective Worker Qualification

In some embodiments, when a prospective worker wishes to work a platform, service, or company that offers gig work opportunities (or similar types of work), a worker can submit an application or create a profile. In some embodiments, the prospective worker can provide personal information, such as name, date of birth, sex, gender identity, location, languages spoken, certifications, experience, and so forth. In some embodiments, personal information can be used to automatically qualify and/or disqualify a prospective worker from certain types of tasks. For example, if a prospective worker indicates that they do not speak a particular language (e.g., Spanish), the prospective worker can be automatically disqualified from performing tasks that require knowledge of the language. As another illustrative example, in the case of recruiting workers to cater an event, a prospective worker can be automatically disqualified if the prospective worker has no experience with the type of cuisine to be prepared and served. As yet another illustrative example, a prospective worker with extensive bartending experience can be automatically qualified to serve drinks at an event. In some embodiments, a system may not be configured to automatically qualify workers. For example, even though a worker indicates experience in a particular area or with a particular task, it can nevertheless be desirable to perform some evaluation of the prospective worker's abilities. For example, a prospective worker who indicates extensive experience serving drinks may have only served beer and wine and thus may not be qualified to prepare and serve mixed drinks. As another example, a prospective worker who indicates experience in conducting diagnostic testing may not have experience or skill with the types of testing to be performed.

Although described above largely in reference to prospective workers, it will be appreciated that it can be desirable to apply similar or the same kinds of evaluation of workers who are active on the platform. Such evaluation can be repeated periodically or continuously. For example, a worker can be required to answer a series of evaluation questions or a worker can have evaluation tasks interspersed with other tasks. Similarly, workers who have ceased completing tasks for a period of time may be re-evaluated when they return to the platform and periodically or continuously thereafter.

In some embodiments, a system can be configured to use eye tracking or presence detection methods while completing tasks, completing evaluations, or both. User presence can be used to determine if the worker or prospective worker is paying attention while completing tasks or evaluations. In some embodiments, user presence detection can be used to determine if a worker or prospective worker is outsourcing work to others such as friends, family, other workers, and so forth. In some embodiments, a system can be configured to capture one or more frames from a camera of a user device of the worker or prospective worker. In some embodiments, the system can compare a captured frame or frames to previously captured frames to ensure that the same worker or prospective worker is each captured frame, each frame is not identical, the worker is facing the camera during capture, and so forth. In some embodiments, image analysis can be used to determine if the worker or prospective worker is looking at the screen during a certain tasks or evaluations. For example, if a worker or prospective worker is proctoring a sample collection phase of a diagnostic test, the system can determine if the worker or prospective worker is watching the sample collection, if the worker or prospective worker is distracting (e.g., reading something else, watching a video, carrying on a conversation, and so forth).

In some embodiments, other information can be captured. For example, in some embodiments, audio can be captured which can indicate, for example, that the worker or prospective worker is carrying on a conversation, watching a video, and so forth. In some embodiments, keyboard inputs, mouse inputs, touch inputs, and/or the like can be used to determine if the worker or prospective worker is distracted. For example, if the worker or prospective worker is typing or providing other inputs during a period when the worker or prospective worker should be observing a testing procedure, the worker or prospective worker may be doing other things besides focusing on the task.

In some embodiments of systems and methods for measuring quality, a worker may be presented with a plurality of questions or seed data to which answers are known. In some embodiments, the worker may be a proctor, an employee, or the public. In some embodiments, the plurality of questions or seed data may be presented to the worker at the start of the worker's employment. In some embodiments, the plurality of questions or seed data may test an accuracy of the worker. The accuracy may be associated with the completion of a specific task. For example, a worker may be presented with an identification (ID) and the worker may be asked whether a person in the ID is the same as the person in a picture presented to the worker.

In some embodiments, at least one of the plurality of questions or seed data may be presented to the worker periodically over a period of time in order to test the accuracy of the worker over a period of time, or a continued accuracy of the worker. In some embodiments, the accuracy of the worker may be compared to a predetermined accuracy threshold. The predetermined accuracy threshold may be a percentage or a number of questions the worker must answer correctly.

In some embodiments, if the accuracy of the worker is below the predetermined accuracy level at any time, the worker may be disciplined. In some embodiments, the worker may be disciplined by removing the worker from a task related to the plurality of questions. In some embodiments, the worker may be disciplined with any other form of discipline or action to remedy the accuracy of the worker.

In some embodiments, an answer to at least one of the plurality of questions may be determined from a plurality of workers. The plurality of workers may form a consensus pool. In some embodiments, a same question may be presented to the plurality of workers. A consensus may be formed when a number of answers reaches a predetermined threshold. In some embodiments, the predetermined threshold may be based on a number of workers in the consensus pool, and a consensus rate. For example, the predetermined threshold may be a predetermined threshold of 99% of 100 workers to form a consensus.

In some embodiments, an average time to answer a question may be measured. A time for a worker to answer a question may be measured while the worker is answering the plurality of questions. The time for the worker to answer the question may be compared to the average time to ensure the worker answers a question in an expected time or answers a plurality of questions in an expected time.

In some embodiments, the worker may be automatically qualified or disqualified from the specific task based on one or more attributes of the worker. For example, a worker may be qualified or disqualified for the specific task based on a language spoken by the worker, a type of cuisine a worker is familiar with, or any other attribute associated with the worker.

In some embodiments, a plurality of tasks may be separated into a plurality of categories. In these embodiments, an accuracy or performance of the worker may be measured for each of the plurality of categories. The accuracy or performance of the worker may be determined by a number of questions the worker answers correctly. The accuracy or performance of the worker may determine a level of compatibility with each of the plurality of categories. In some embodiments, the level of compatibility may be used to determine which of the plurality of tasks the worker may be assigned to. If a worker has a higher level of compatibility with one of the plurality of categories, the worker may be assigned to tasks in the one of the plurality of categories more often.

In some embodiments, the system may use eye tracking or other features indicative of user presence to determine if the worker is paying attention while completing tasks or the plurality of questions. The user presence features may also determine if the worker is outsourcing work to other workers. For example, the system may periodically capture a frame of a video recording. In some embodiments, the system may compare a captured frame to previously captured frames to ensure a same worker is in every captured frame, each frame is not identical, and/or that the worker is facing a camera capturing the video recording.

In some embodiments, the systems, methods, and devices described herein may be used to gather data for a machine learning algorithm. For example, the worker may be presented with a picture, and the worker may be asked one or more questions about the picture. In this way, the systems and methods described herein may be used to label data for a machine learning algorithm. In some embodiments, the one or more questions may have discrete answers, such as yes or no answers. In some embodiments, the data may be associated with a task that may not be automated, such as ID matching, or determining the contents of a photograph.

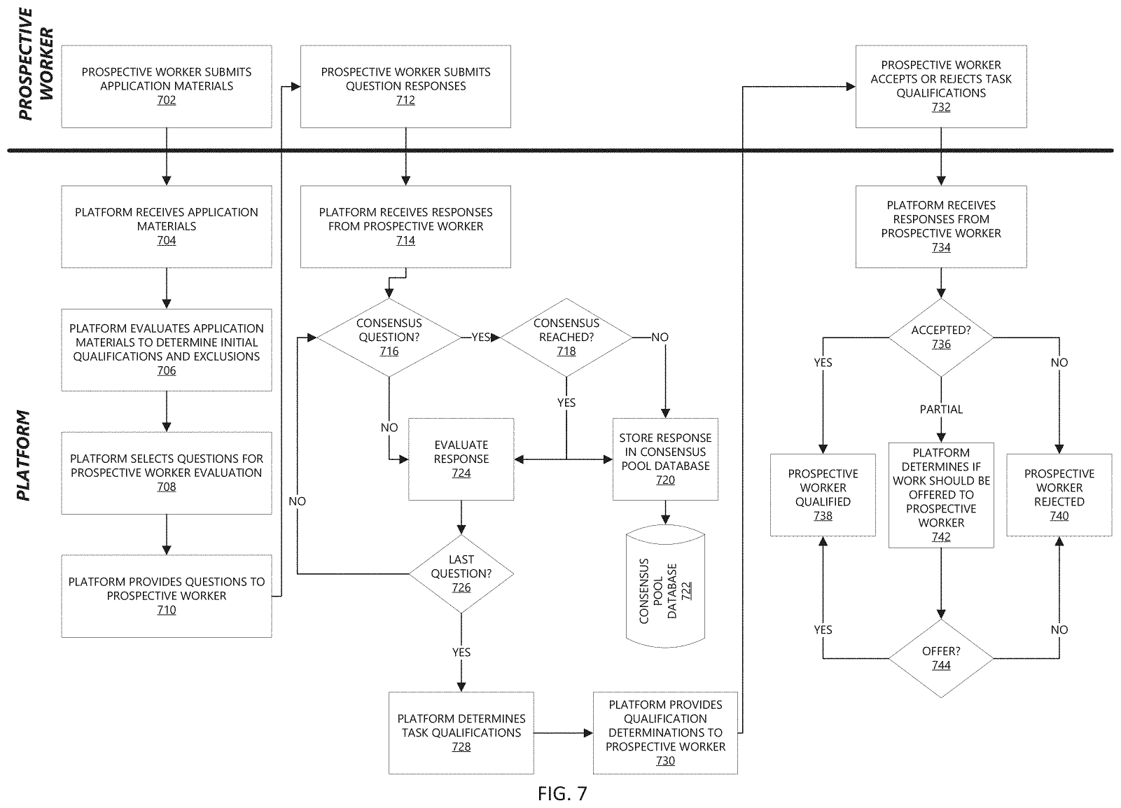

FIG. 7 is a block diagram that illustrates an example process for determining qualifications of a prospective worker according to some embodiments. At block 702, a prospective worker can submit application materials to a platform. The application materials can include, for example, identifying information (e.g., name, date of birth, social security number, driver's license number, and so forth), address, skills, qualifications, certifications, languages spoken (which can include a competency level), and so forth. At block 704, the platform can receive the application materials submitted by the prospective worker. At block 706, the platform can evaluate the application materials to determine initial qualifications and/or exclusions. For example, if a prospective worker is located in a country or region where the platform does not operate, the prospective worker can be rejected. If the worker indicates proficiency or lack or proficiency with a language, the worker can be initially qualified or can be disqualified from performing tasks that require knowledge of the language. At block 708, the platform can select questions to use in evaluating the prospective worker. For example, the questions can be stored in a question database and can be selected at random, based on the application materials (e.g., if a prospective worker indicates certification or experience in performing drug testing, the prospective worker can be provided with questions related to drug testing). At block 712, the worker can submit responses to the questions. At block 714, the platform can receive the responses from the prospective worker. As discussed above, in some embodiments, there can be only questions with clear answers. In some embodiments, there can be questions whose answers are determined by consensus. In some embodiments, users can be asked a combination of clear answer and consensus questions. For each question asked to the prospective worker, the platform can, at decision 716, determine if the question was a consensus-based question. If not, the system can evaluate the response at block 724. If the question is a consensus question, then at decision 718, the platform can determine if a consensus has been reached. If not, the system can, at block 720 store the prospective worker's response in a consensus pool database 722. If consensus has been beached the system can store in the prospective worker's response in the consensus pool database 722 at block 720. As discussed above, in some embodiments, the system can perform one or more checks to determine if the prospective worker's response should be included in the consensus pool database 722. For example, if the prospective worker has answered other questions in a way that indicates the prospective is highly unqualified or that the prospective worker has not answered questions in a serious manner (e.g., has randomly selected answers, has intentionally selected incorrect answers, or didn't spend enough time on each question to have actually read the questions, that prospective worker's responses may not be used to build the consensus pool.

At decision 726, the platform can determine if it has evaluated all questions answered by the prospective worker. If not, the process can continue from decision 716 and can repeat until all question responses have been processed.

At block 728, the platform can determine tasks qualifications. The determination can be based at least in part on the prospective worker's responses to the questions. In some embodiments, other factors can be considered, such as language, location, and so forth. For example, the platform can consider information in the application materials submitted by the prospective worker. In some embodiments, a worker who was initially qualified at block 706 can be qualified or disqualified from a task, type of task, language, and so forth at block 728. For example, a user who indicated Spanish proficiency on the application materials and was initially qualified to conduct tasks in Spanish at block 706 can be disqualified at block 728 from conducting tasks in Spanish if the prospective worker's responses indicate a lack of adequate proficiency in Spanish. Similarly, if a prospective worker indicated some knowledge of Spanish on the application materials, for example, the prospective worker can be qualified to conduct Spanish-language tasks if the prospective worker's responses indicate adequate proficiency in the language.

In some embodiments, the process can stop at block 728. In other embodiments, the process can continue. For example, at block 730, the platform can inform the user of the qualification determinations. At block 732, the prospective worker can review the qualifications and, in some embodiments, can accept or reject the determinations. In some embodiments, the prospective worker may be able to selectively accept and reject individual qualifications or groups of qualifications. For example, if a prospective worker's results indicate they are qualified to perform throat swab testing, but the prospective worker doesn't feel comfortable or confident in doing such work, in some embodiments the prospective worker could opt not to perform that type of task. In some embodiments, prospective workers may not be given such options and may only be able to accept or reject the platform's results in total.

At block 734, the platform can receive a response or responses from the prospective work. At decision 736, if the prospective worker has rejected the qualifications, the platform can reject the prospective worker at block 740. If the prospective worker has accepted the qualifications, the prospective worker can be qualified at block 738. The prospective worker can then move onto additional steps in an onboarding process. For example, if the prospective worker hasn't already provided tax information, proof of work authorization, and the like, the platform can collect such information from the prospective worker. In some embodiments, the prospective worker can partially accept and partially reject the qualifications. At block 742, the platform can determine if work should be offered to the prospective worker. For example, if the prospective worker has opted out of only a few types of tasks or uncommon tasks, the prospective worker can likely still be offered work. However, if the prospective worker has opted out of many types of tasks, there may be insufficient work available for the prospective worker or it may otherwise be undesirable to assign work to the prospective worker. At decision 744, the platform cannot offer work to the prospective worker, in which case the prospective work can be rejected at block 740. If, at decision 744, the platform offers work to the prospective worker, the prospective worker can be qualified at block 738.

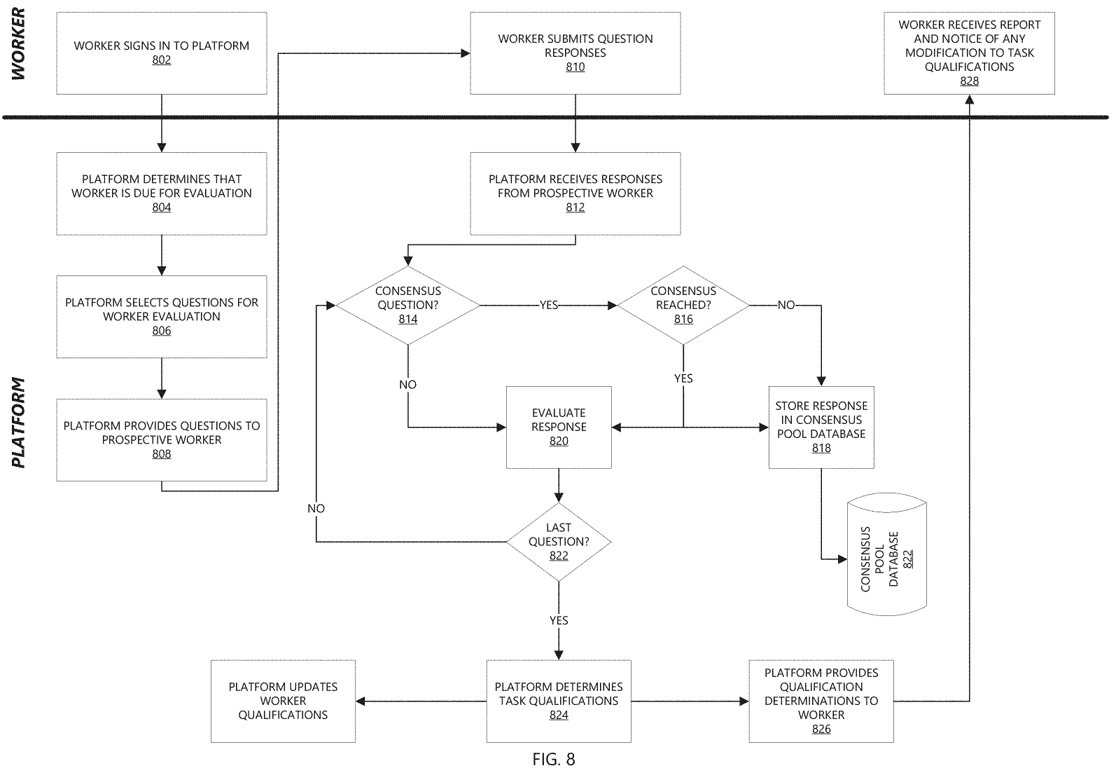

In some embodiments, the process shown in FIG. 8 can be adapted to existing workers. At block 812, the worker can submit responses to the questions. At block 802, the existing worker signs into the platform. At block 804, the platform may determine whether or not the worker is due for evaluation. At block 806, the platform can select questions to use in evaluating the existing worker. For example, the questions can be stored in a question database and can be selected at random, based on the application materials (e.g., if a prospective worker indicates certification or experience in performing drug testing, the prospective worker can be provided with questions related to drug testing). The platform may provide the selected questions to the user at block 808. At block 810, the worker can submit responses to the questions. At block 814, the platform can receive the responses from the worker. As discussed above, in some embodiments, there can be only questions with clear answers. In some embodiments, there can be questions whose answers are determined by consensus. In some embodiments, users can be asked a combination of clear answer and consensus questions. For each question asked to the existing worker, the platform can, at decision 714, determine if the question was a consensus-based question. If not, the system can evaluate the response at block 820. If the question is a consensus question, then at decision 816, the platform can determine if a consensus has been reached. If not, the system can, at block 818 store the existing worker's response in a consensus pool database 822. If consensus has been reached the system can store in the existing worker's response in the consensus pool database 822 at block 720. As discussed above, in existing embodiments, the system can perform one or more checks to determine if the existing worker's response should be included in the consensus pool database 822. For example, if the existing worker has answered other questions in a way that indicates the prospective is highly unqualified or that the existing worker has not answered questions in a serious manner (e.g., has randomly selected answers, has intentionally selected incorrect answers, or didn't spend enough time on each question to have actually read the questions, that existing worker's responses may not be used to build the consensus pool.

At decision 822, the platform can determine if it has evaluated all questions answered by the prospective worker. If not, the process can continue from decision 814 and can repeat until all question responses have been processed.

At block 824, the platform can determine tasks qualifications. The determination can be based at least in part on the existing worker's responses to the questions. In some embodiments, other factors can be considered, such as language, location, and so forth. In some embodiments, the process can update worker qualifications and stop at block 824. In other embodiments, the process can continue. For example, at block 826, the platform can inform the user of the qualification determinations. At block 828, the existing worker may receive an report and notice of any modification to task qualifications.

As described above, in some embodiments, it can be desirable to perform additional actions while evaluating workers or prospective workers. For example, some remotely-located workers or prospective workers may attempt to cheat, for example by looking up by seeking input from someone else. In some cases, workers or prospective workers may attempt to look up answers, such as looking up how to interpret a lateral flow test for a particular infectious disease. Accordingly, in some embodiments, it can be advantageous to implement monitoring procedures during the evaluation of workers or prospective workers. If suspicious activity is detecting, various actions can be taken.

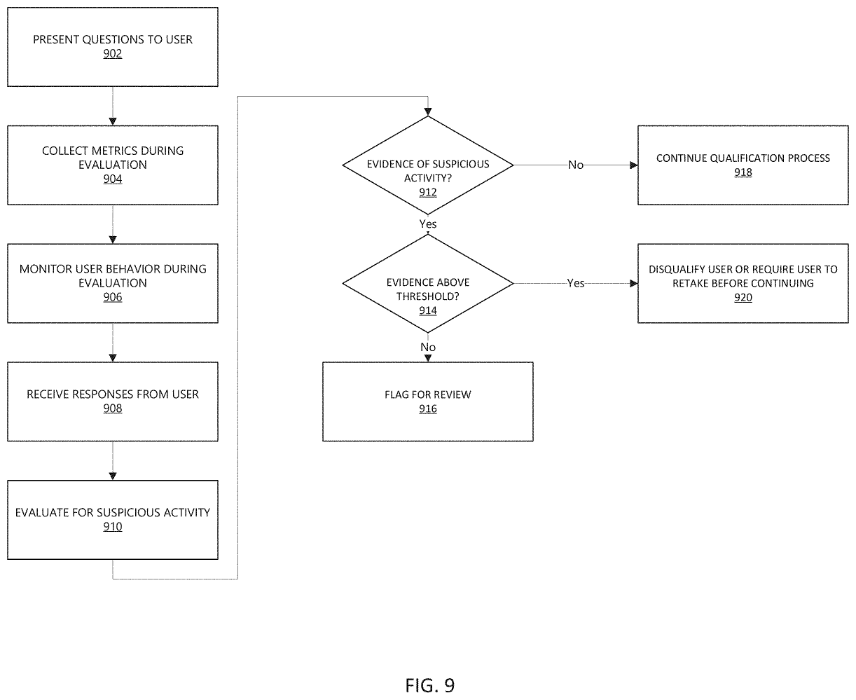

FIG. 9 is a block diagram that illustrates an example process for evaluating workers or prospective workers according to some embodiments. At block 902, the platform can present questions to the user. At block 904, the platform can collect various metrics during the evaluation procedure. For example, metrics could include time per question, delay between reading a question and picking or inputting a response, and so forth. The platform can, at block 906, monitor using behavior during the evaluation procedure. For example, the platform can monitor keyboard inputs, mouse inputs, change in window focus, and so forth. Such events can be indicative of issues such as consulting web resources, chatting with another person, sharing a screen with another person, and so forth. In some embodiments, the platform can monitor the worker or prospective worker using other input devices a user device, such as a camera, microphone, or the like. In some embodiments, the platform can monitor for speaking, glancing away from the screen, and so forth. For example, the platform may be configured to detect that the worker or prospective worker's gaze has drifted away for a period of time that may indicate that the worker or prospective worker is consulting another source, is having someone else complete the evaluation, and so forth. For example, if the worker or prospective worker is responding to questions while not looking at the screen, the platform can be configured to identify a high likelihood of cheating or other suspicious behavior. In some embodiments, audio capture can be used to detect that a user is engaging in a discussion with another individual. For example, a person being evaluated can be getting hints or other guidance from someone out of the field of view of the camera.

At block 908, the platform can receive responses from the user. At block 910, the platform can evaluate the session for evidence of suspicious activity. In some embodiments, suspicious activity can be determined solely based on responses to questions, solely based on monitored behavior, or solely based on collected metrics (e.g., a worker or prospective worker who rapidly answers each question correctly may be engaged in cheating). In some embodiments, any combination of metrics, question responses, and/or monitored user behavior can be used in evaluating the session for suspicious activity.

At decision 912, if there is no evidence of suspicious activity, the platform can, at block 918, continue a qualification process or other process. For example, the platform can continue a process such as the processes depicted in FIG. 1 and FIG. 2.

If, at decision 912, suspicious activity is detected, at decision 914, the platform can determine if the evidence is above a threshold value. For example, a suspicious activity score, sufficient activity threshold, etc., can be defined by administrators of the system. Over time, a suspicious activity threshold can be adjusted automatically. In some cases, a platform can be configured so that the threshold is zero (e.g., if any suspicious activity is detected, the result is invalidated). In some embodiments, if the evidence is above the threshold, the platform can disqualify the user or require the user to retake the evaluation before continuing at block 920. If evidence is below the threshold but still present, the platform can flag the session for review at block 916.

### Proctor Management on Remote Diagnostic Testing Platforms

In some embodiments, a management system may allow for one or more proctors to connect to a user over a network, for example, by way of a proctored examination platform. In some embodiments, a proctor may connect to the network with a proctor computing device. For example, the proctor computing device may be a computer, a mobile computing system, or any other type of computing system. In some embodiments the user may connect to the network with a user computing device. For example, the user computing device may be a computer, a mobile computing system, or any other type of computing system.

In some embodiments, the management system may comprise a video conferencing system. In some embodiments, the video conferencing system may be configured to facilitate video calls, audio calls, or telemedicine calls. In some embodiments, the management system may connect the user to the video conferencing system. In some embodiments, the management system may request user authentication prior to connecting the user with the proctor. In some embodiments, the system may connect one or more proctors to the video conferencing system. In some embodiments, the one or more proctors may connect to the video conferencing system with a customer service connection system. In some embodiments, multiple proctors may connect to the video conferencing system at a same time.

In some embodiments the one or more proctors may leave the video conferencing system. In some embodiments, the one or more proctors may connect to and/or leave the video conferencing session one or more times during one video call or telemedicine visit. In some embodiments, the one or more proctors may connect to and/or leave the video conferencing session without the video conferencing system notifying the user.

In some embodiments, a video of the proctor may be a video recorded from the proctor device. In some embodiments, the video of the proctor may be a computer graphics representation of a proctor. In some embodiments, the computer graphics representation may be the same computer graphics representation during the entire call.

In some embodiments, the one or more proctors and the user may communicate through audio. In some embodiments, a voice of each of the one or more proctors may be altered or manipulated such that the user hears a same voice when each of the one or more proctors communicates through audio.

In some embodiments, the system may include a recording module or system. In some embodiments the recording module or system may automatically record the video conferencing system. The recording module or system may record the video conferencing system when a user joins, when one of the one or more proctors joins, or at any time during the video call or telemedicine call.

In some embodiments, the management system may include multiple proctors. In some embodiments, each of the multiple proctors may include a set of one or more skills. In some embodiments, the set of one or more skills may include, a language, a test type, a test stage, geographic location of a proctor, an internet strength of a proctor, a latency of a proctor, a special customer request, a training level of a proctor and/or any other skill.

In some embodiments, the special customer request may include a request from a customer including a company, airline, a government entity, or any other customer, that a certain number of proctors connect with the customer's users. In some embodiments, the customer may request more proctors or less proctors depending on how many of the customer's users are using the management system at a same time. In some embodiments, the special customer request may include a geographic location of the proctor. In some embodiments, the special customer request may include a screen resolution of the proctor computing device. In some embodiments, the special customer request may include a request that the proctor is isolated.

In some embodiments, each of the multiple proctors may have a queue or waiting room associated with the set of one or more skills associated with each proctor. In some embodiments, multiple proctors may have the same set of one or more skills. In some embodiments, each queue may be associated with multiple proctors. In some embodiments, the queue or waiting room may be associated with multiple proctors. In some embodiments, each queue may include one or more users that require the set of one or more skills associated with the queue or waiting room.

In some embodiments, one or more proctors may connect with a next user in the queue or waiting room. In some embodiments, an order of users in the queue or waiting room may be determined by how long a user has been waiting or any other factor commonly used for determining an order of a queue or waiting room. In some embodiments, a manager or agent may select which user each proctor connects with.

In some embodiments, each of the multiple proctors may have a routing profile associated with the set of one or more skills associated with each proctor. In some embodiments, multiple proctors may have the same routing profile. In some embodiments, the routing profile may include one or more queues or waiting rooms associated with the set of one or more skills associated with each proctor. For example, the routing profile of a proctor may have a set of skills bnc_step1, bnc_step2, English, Spanish, and the queues or waiting that include only skills in the set of skills.

In some embodiments, one or more skills may include a priority level. In some embodiments, the priority level may be used to order queues or waiting rooms in each routing profile. In some embodiments, queues with high priority skills may be treated preferentially by the management system. In some embodiments, queues may be sorted in an order. In some embodiments, the order may be determined by how similar the set of one or more skills associated with a proctor is to a set of one or more skills associated with a queue or waiting room. The order may determine which queue or waiting room the proctor connects to users from first. In some embodiments, if a first queue or waiting room is empty, or a wait time of the first queue or waiting room is less than a predetermined time, the proctor may connect to users in a second queue or waiting room. In some embodiments, the first queue or waiting room may be the queue or waiting room with a set of one or more skills that match the set of one or more skills associated with the proctor. In some embodiments, the second queue or waiting room may be a next queue or waiting room, or a queue or waiting room with a less preferential set of one or more skills. In some embodiments, the proctor may select an order of the queues.

In some embodiments, queues or waiting rooms may include a name. In some embodiments, the name may have a maximum length of 127 characters. In some embodiments, queues or waiting rooms may include a description. In some embodiments, the description may have a maximum length of 250 characters. In some embodiments, routing profiles may include a name. In some embodiments, the name may have a maximum length of 127 characters. In some embodiments, routing profiles may include a description. In some embodiments, the routing profiles may have a maximum length of 250 characters.

In some embodiments, names and descriptions of queues or waiting rooms and routing profiles may be stored on a memory of the computing system. In some embodiments, the names and descriptions may be displayed on the proctor computing device. In some embodiments, the names and descriptions may be recalled for the memory. In some embodiments, a hash algorithm may be used to store and recall the names and descriptions.

In some embodiments, a proctor may sign into the management system. In some embodiments, when a proctor signs in, the proctor may have a profile. The profile may include a set of one or more skills associated with the proctor. In some embodiments, the management system may search for a queue and/or a routing profile that corresponds to the set of one or more skills associated with the profile of the proctor. In some embodiments, if the queue and/or routing profile exists, the proctor may be added to the routing profile containing the queue.

In some embodiments, if the queue and/or routing profile does not exist, the management system may create a queue and a routing profile that are associated with the set of one or more skills associated with the profile of the proctor. In some embodiments, the created queue and routing profile may have a skill set that matches the set of one or more skills associated with the proctor. In some embodiments, the proctor may be attached to the routing profile and the queue may be added to the routing profile.

In some embodiments, the routing profile may only contain queues that correspond to the set of one or more skills associated with the profile of the proctor and the routing profile.

In some embodiments, the management system will search for queues that contain at least one skill of the set of one or more skills associated with the proctor. In some embodiments, the queues may be added to the routing profile associated with the profile of the proctor if the queue only contains skills in the set of one or more skills associated with the proctor.

In some embodiments, a user may connect to the management system. In some embodiments, when the user connects to the management system, the user may be prompted to request a set of skills. In some embodiments, based on a requested set of skills, the management system may search for a queue associated with the exact requested set of skills. If the queue associated with the exact requested set of skills exists, the management system may as the user to the queue. In some embodiments, if the queue associated with the exact requested set of skills does not exist, the management system may search for queues that include at least the requested set of skills and add the user to one of the queues.

In some embodiments, the management system may automatically and dynamically add a user to an optimal queue. In some embodiments, the optimal queue may be a queue with a shortest wait time. In some embodiments, the optimal queue may be a queue with the most available proctors. In some embodiments, the optimal queue may be a queue with the least number of skills other than the requested set of skills. In some embodiments the user may be added to more than one queue.

In some embodiments, the management system may include third party developed systems. For example, the video conferencing system may be developed by a third party. In some embodiments, the entire management system may be developed by a third party. In some embodiments, preexisting management systems may be updated to provide features of the embodiments described above. In some embodiments at least a portion of the management system may be custom made. In some embodiments, Simple Notification Service (SNS) may be used as the video conferencing system. In some embodiments, Simple Queue Service (SQS) may be used to create queues.

### Proctor Management

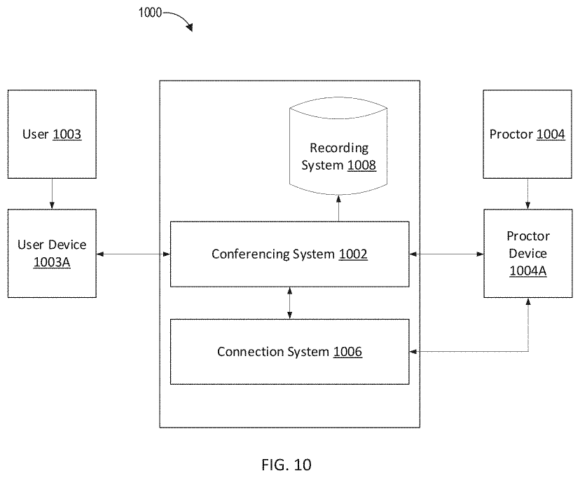

FIG. 10 shows a schematic of a management system 1000. In some embodiments, the management system 1000 can include a conferencing system or module 1002. In some embodiments, the conferencing system 1002 can be configured to connect a user 1003 and a proctor 1004 to a telehealth session. In some embodiments, the conferencing system 1002 can be configured to connect the user 1003 and the proctor 1004 via a video conferencing session. In some embodiments, the conferencing system 1002 can facilitate connection of the user 1003 with the proctor 1004 via live video (e.g., over the Internet of cellular communication network). In some embodiments, the conferencing system 1002 can be configured to facilitate video calls, audio calls and/or telemedicine calls. In some embodiments, the user 1003 may access the conferencing system 1002 via a user device 1003A. In some embodiments, the proctor 1004 may access the conferencing system 1002 via a proctor device 1004A.

In some embodiments, the conferencing system 1002 can request user authentication prior to connecting the user 1003 to a proctor 1004. In some embodiments, user authentication can include the user 1003 logging into a user profile, the user 1003 entering a password, the user 1003 providing a biometric identifier, the user 1003 capturing an image of the user 1003, the user 1003 capturing an image of an ID of the user 1003, and/or any other user authentication.

In some embodiments, the conferencing system 1002 can connect one or more proctors 1004 to the video conferencing session. In some embodiments, the conferencing system 1002 can connect a first proctor 1004 to the video conferencing session for a first portion of the video conferencing session and connect a second proctor 1004 to the video conferencing session for a second portion of the video conferencing session.

In some embodiments, a proctor 1004 can leave the video conferencing session after a portion of the video conferencing session and/or a diagnostic test that the proctor 1004 is assigned to proctor. In some embodiments, the proctor 1004 can join and/or leave the video conferencing session one or more times during the video conferencing session. In some embodiments, the proctor 1004 can join and/or leave the video conferencing session 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 30 times, 40 times, 50 times, 60 times, 70 times, 80 times, 90 times, 100 times, 150 times, 200 times, 250 times, 300 times, 350 times, 400 times, 450 times, 500 times, and/or any value between the aforementioned values.

In some embodiments, the proctor 1004 can connect to and/or leave the video conferencing session without the user 1003 knowing and/or without the conferencing system 1002 sending a notification to the user 1003 that the proctor 1004 is joining and/or leaving the video conferencing session.

In some embodiments, a video of the proctor 1004 that the conferencing system 1002 can display to the user 1003 can include a video recorded by a camera of the proctor device. In some embodiments, a video of the proctor 1004 that the conferencing system 1002 can display to the user 1003 can include a computer graphics representation of a proctor 1004. In some embodiments, the computer graphics representation can include a same computer graphics representation during the call. In some embodiments, the computer graphics representation can remain a same computer graphics representation when a first proctor 1004 leaves the video conferencing session and a second proctor 1004 joins the video conferencing session.

In some embodiments, the user 1003 can communicate with the one or more proctors 1004 via audio signals captured by the user device. In some embodiments, the one or more proctors 1004 can communicate the user 1003 via audio signals captured by proctor devices. In some embodiments, the audio signals captured by the proctor devices can be manipulated and/or modified so the user device plays a same voice to the user 1003 when each of the one or more proctors communicate with the user 1003 via audio. In some embodiments, the audio signals captured by the proctor devices can be manipulated and/or modified so each proctor sounds the same to the user 1003. In some embodiments, playing a same voice to the user 1003 through the video conferencing session can allow the proctor management system 1000 to efficiently use proctors' time while maintaining perceived seamlessness for the user 1003.

In some embodiments, the proctor management system 1000 can include a recording system or module 1008. In some embodiments, the recording system 1008 can be configured to automatically and/or dynamically record at least a portion of a video conferencing session. In some embodiments, the recording system 1008 can record the entire video conferencing session. In some embodiments, the recording system 1008 can be configured to automatically and/or dynamically start the recording of at least the portion of the video conferencing session at the start of the video conferencing session. In some embodiments, the recording system 1008 can be configured to automatically and/or dynamically start a recording of at least the portion of the video conferencing session when the user 1003 joins that video conferencing session. In some embodiments, the recording system 1008 can be configured to automatically and/or dynamically start the recording of at least the portion of the video conferencing session when a proctor 1004 joins the video conferencing session. In some embodiments, the recording system 1008 can automatically and/or dynamically end the recording of at least the portion of the video conferencing session when the proctor 1004 leaves the video conferencing session. In some embodiments, the recording system 1008 can end the recording of at least the portion of the video conferencing session when the video conferencing session ends.

In some embodiments, the recording system 1008 can transmit video data of the recording of at least the portion of the video conferencing session to a storage system or server. In some embodiments, the storage system or server can store the video data so that the recording of at least the portion of the video conferencing session can be accessed at a later time. In some embodiments, one or more portions of a recording of a video conferencing session can be transmitted to a proctor 1004 that will join a subsequent portion of the video conferencing session. In some embodiments, the proctor 1004 can review and/or analyze the one or more portions of the recording of the video conferencing session to determine background information for the video conferencing session, such as, previous events during the video conferencing session, comments, questions, problems, abnormalities, or the like that occurred during the video conferencing session before the proctor 1004 joins the subsequent portion of the video conferencing session.

In some embodiments, the recording system 1008 and/or the storage system can automatically and/or dynamically add tags to stored video data. In some embodiments, the tags can include a session identifier, a test type, and/or other metadata. In some embodiments, the tags can include only deidentified information. In some embodiments, the tags the management system 1000 and/or a telehealth platform can use the tags for statistical analysis, research programs, and/or quality improvement.

In some embodiments, the management system 1000 can include a connection system 1006. In some embodiments, the connection system 1006 can be configured to determine which proctor 1004 to connect with the user 1003 via the conferencing system 1002. In some embodiments, the connection system 1006 can be configured to determine which user 1003 to connect with the proctor 1004.

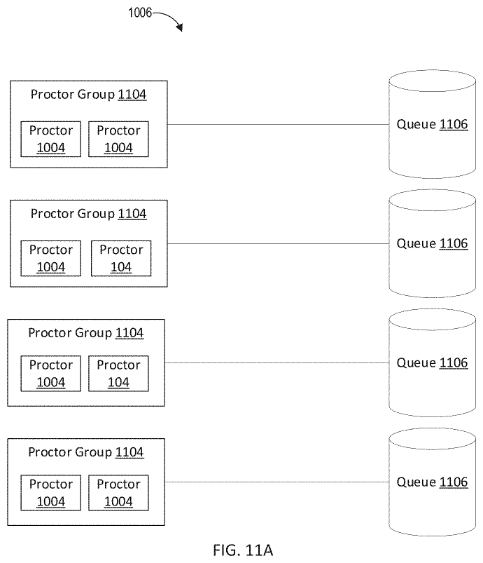

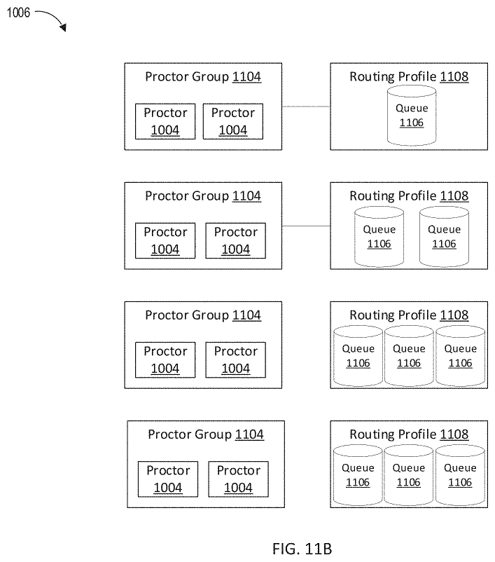

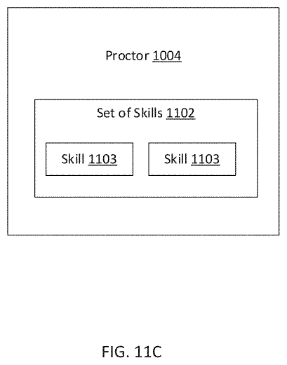

FIG. 11A shows an embodiment of a connection system 1006. In some embodiments, the connection system 1006 can select a proctor 1004 from a plurality of proctors 1004. As shown in FIG. 11C, in some embodiments, each proctor 1004 can include a set of skills 1102. In some embodiments, the set of skills 1102 can include skills 1103. In some embodiments, the set of skills 1102 can include one or more skills 1103. In some embodiments, the set of skills 1102 can include two or more skills 1103. In some embodiments, the set of skills 1103 can include 1 skill 1103, 2 skills 1103, 3 skills 1103, 4 skills 1103, 5 skills 1103, 6 skills 1103, 7 skills 1103, 8 skills 1103, 9 skills 1103, 10 skills 1103, 11 skills 1103, 12 skills 1103, 13 skills 1103, 14 skills 1103, 15 skills 1103, 16 skills 1103, 17 skills 1103, 18 skills 1103, 19 skills 1103, 20 skills 1103, 21 skills 1103, 22 skills 1103, 23 skills 1103, 24 skills 1103, 25 skills 1103, 26 skills 1103, 27 skills 1103, 28 skills 1103, 29 skills 1103, 30 skills 1103, 31 skills 1103, 32 skills 1103, 33 skills 1103, 34 skills 1103, 35 skills 1103, 36 skills 1103, 37 skills 1103, 38 skills 1103, 39 skills 1103, 40 skills 1103, 41 skills 1103, 42 skills 1103, 43 skills 1103, 44 skills 1103, 45 skills 1103, 46 skills 1103, 47 skills 1103, 48 skills 1103, 49 skills 1103, 50 skills 1103, and/or more skills 1103. In some embodiments, each proctor 1004 can include a set of skills 1102 with a same number of skills 1103. In some embodiments, one or more proctors 1004 can include a set of skills 1102 with a different number of skills 1103.

In some embodiments, the skills 1103 can include one or more languages spoken by the proctor 1004. In some embodiments, the skills 1103 can include one or more test types the proctor 1004 is certified to proctor a telehealth session for. For example, in some embodiments, a proctor 1004 may be designated and/or certified to administer medical screenings, medical diagnostic test, and/or non-medical screenings (e.g., pre-employment, pre-certification, random screening, etc.). In some embodiments, the one or more test types can include a test manufacturer, a condition the test is configured to detect, a sample collection method, a stage of testing procedure, and/or any other test type. In some embodiments, the skills 1103 can include a geographic location of the proctor 1004. For example, a city, state, country, or other geographic region may require the proctor 1004 have particular certifications or qualifications, or particular telehealth session requirements. In some embodiments, the skills 1103 can include an internet speed or strength of the proctor 1004, and/or a latency of the proctor 1004. In some embodiments, the set of skills 1004 can include a user classification assigned to the proctor 1004 and/or a user classification the proctor 1004 is certified to proctor a telehealth session for. In some embodiments, the user classification can include a VIP status of a user, an employer of a user, whether a user is a government employee, an age of a user, an experience level of a user, a reason why a user is taking the test, a vendor of the test, and/or any other user classification. In some embodiments, the vendor of the test may include an airline, an insurance group, a government entity, and/or any other organization or entity requiring a user to take the test. In some embodiments, the skills 1103 can include a training or certification level of a proctor 1004.

In some embodiments, each proctor 1004 of the plurality of proctors 1004 can include a different set of skills 1102. In some embodiments, two or more proctors 1004 of the plurality of proctors 1004 can include a same set of skills 1102.

In some embodiments, the connection system 1006 can include one or more proctor groups 1104. In some embodiments, each proctor group 1104 of the one or more proctor groups 1104 can include one or more proctors 1004. In some embodiments, one or more of the proctor groups 1104 of the one or more proctor groups 1104 can include no proctors 1004.

In some embodiments, each proctor group 1104 can correspond to a particular set of skills 1102. In some embodiments, each proctor group 1104 can include one or more proctors 1004 with the particular set of skills 1102. In some embodiments, each proctor group 1104 can include every proctor 1004 with the particular set of skills 1102. For example, a first proctor group 1104 can include proctors 1004 with a first set of skills 1102 that includes a first skill 1103 and a second skill 1103, and a second proctor group 1104 can include proctors 1004 with a second set of skills 1102 that includes a third skill 1103 and a fourth skill 1103. In some embodiments, each proctor group 1104 can include one or more proctors 1004 with the same set of skills 1102 as the particular set of skills 1102 that corresponds to the proctor group 1104. In some embodiments, each proctor group 1104 can include every proctor 1004 with the same set of skills 1102 as the particular set of skills 1102 that correspond to the proctor group 1104.

In some embodiments, the connection system 1006 can include a plurality of queues 1106. In some embodiments, each proctor group 1104 can include one queue 1106 of the plurality of queues 1106. In some embodiments, each queue 1106 can include one or more users 1003 waiting to connect with a proctor 1004 with the particular set of skills 1102 that corresponds to the proctor group 1104.

In some embodiments, the connection system 1006 can connect a first user 1003 in a queue 1106 with an available proctor 1004 of the proctor group 1104 that includes the queue 1106. In some embodiments, if no proctor 1004 of the proctor group 1104 that includes the queue 1106 is available the connection system 1006 can connect the first user 1003 in the queue 1106 when a proctor 1004 is available.

FIG. 11B shows another embodiment of a connection system 1006. In some embodiments, each proctor group 1104 can include a routing profile 1108. In some embodiments, the routing profile 1108 can include one or more queues 1106. In some embodiments, the routing profile 1108 of each proctor group 1104 can include the queue 1106 of the proctor group 1104, as described above with reference to FIG. 11A.

In some embodiments, the routing profile 1108 of each proctor group 1104 can include one or more queues 1106 of other proctor groups 1104 of the one or more proctor groups 1104. In some embodiments, the routing profile 1108 of a proctor group 1104 can include the queues 1106 of one or more other proctor groups 1104 wherein the particular set of skills 1102 that corresponds to the proctor group 1104 includes every skill 1103 of the set of skills 1102 of the one or more other proctor groups 1104. For example, in some embodiments, the set of skills 1102 of the first proctor group 1104 can include a first skill 1103, a second skill 1103, and a third skill 1103, and the routing profile 1108 of the first proctor group 1104 can include a queue 1106 of a second proctor group 1104 wherein the set of skills 1102 of the first proctor group 1104 includes the first skill 1103 and the second skill 1103, and the routing profile 1108 of the first proctor group 1104 can include a queue 1106 of a third proctor group 1104 wherein the set of skills 1102 of the third proctor group 1104 includes the second skill 1103 and the third skill 1103.

In some embodiments, the routing profile 1108 of each proctor group 1104 can include the queue 1106 of every other proctor group 1104 wherein the particular set of skills 1102 that corresponds to the proctor group 1104 includes every skill 1103 of the set of skills 1102 of the one or more other proctor groups 1104.

In some embodiments, the connection system 1006 can connect an available proctor 1004 of the proctor group 1104 to a first user 1003 in any of the queues 1106 of the routing profile 1108 of the proctor group 1104. In some embodiments, the queues 1106 of the routing profile 1108 can be sorted in an order based on a priority level of each queue 1106 of the routing profile 1108. In some embodiments, the connection system 1006 can connect the proctor 1004 to users 1003 of a queue 1106 with a highest priority level first. In some embodiments, if the queue 1106 is empty (i.e., no users 1003 are in the queue 1106), the connection system 1006 can connect the proctor 1004 to users 1003 of a queue 1106 with a next highest priority level. In some embodiments, the connection system 1006 can connect the proctor 1004 to users 1003 of a queue 1106 with the highest priority level that is not empty (i.e., includes one or more users 1003).

In some embodiments, the queue 1106 of the proctor group 1104 can include the highest priority level. In some embodiments, one or more skills 1103 of the set of skills 1102 of the other proctor groups 1104 can include a high priority level. In some embodiments, the order of the one or more queues 1106 of the other proctor groups 1104 can be based on a number of skills 1103 of the set of skills 1102 of the other proctor groups 1104 that include a high priority level. For example, a set of skills 1102 of a second proctor group 1104 can include two skills 1103 with a high priority level, and the second proctor group 1104 can include a higher priority level than a third proctor group 1104 that includes a set of skills 20C including one skill 1103 with a high priority level. In some embodiments, the order of the one or more queues 1106 of the other proctor groups 1104 can be based on a similarity between the set of skills 1102 of the proctor group 1104 and the set of skills 1102 of the other proctor groups 1104. In some embodiments, the queues 1106 of the other proctor groups 1104 with the fewest number of skills 1103 of the set of skills 1102 that are different from the skills 1103 of the set of skills 1102 of the proctor group 1104 of the routing profile 1108 can include a higher priority level. For example, in some embodiments, the queue 1106 of a second proctor group 1104 with one different skill 1103 from a first proctor group 1104 can include a higher priority level than the queue 1106 of the third proctor group 1104 with two different skills 1103 from the first proctor group 1104. In some embodiments, queues 1106 that include more users 1003 can include a higher priority. In some embodiments, queues 1106 that include a longer wait time can include a higher priority. In some embodiments, the connection system 1006 can automatically and dynamically update the priority level of each queue 1106 based on a number of proctors 1004 in a proctor group 1104, a number of available proctors 1004 in a proctor group 1104, a number of available proctors 1004 in one or more other proctor groups 1104, a current wait time of each queue 1106, and/or a number of users 1003 in each queue 1106.

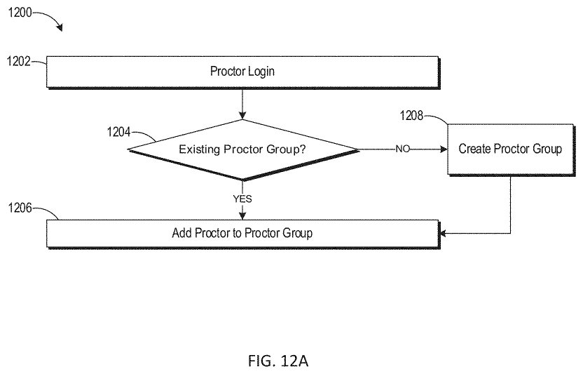

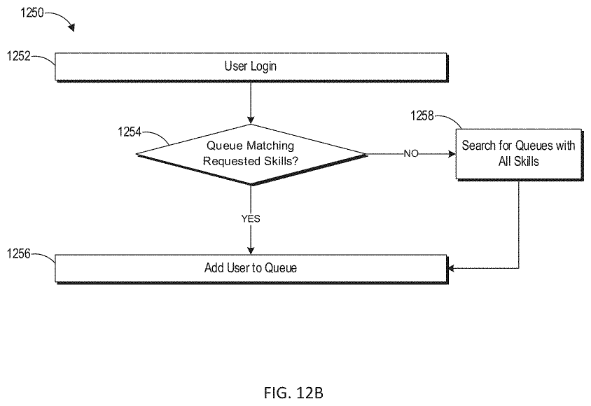

FIG. 12A shows a flowchart of a method 1200 for adding a proctor 1004 to a proctor group 1104. In some embodiments, at step 1202, the proctor 1004 can login to the management system 1000, the connection system 1006 and/or a telehealth platform. In some embodiments, the management system 1000, the connection system 1006 and/or the telehealth platform can include a proctor profile. In some embodiments, the proctor profile can include proctor information. In some embodiments, the proctor information can include identity information, such as, for example, a name of the proctor 1004, an identification number of the proctor 1004, a certification status of the proctor 1004, the set of skills 1102 of the proctor 1004, and/or any other information associated with the proctor 1004.

In some embodiments, at step 1204, the connection system 1006 can search for a proctor group 1104 that includes a same set of skills 1102 as the set of skills 1102 of the proctor profile. In some embodiments, if the connection system 1006 identifies a proctor group 1104 that includes the same set of skills 1102 as the set of skills 1102 of the proctor profile, at step 1206, the system 1006 can add the proctor 1004 to the proctor group 1104.

In some embodiments, if the connection system 1006 cannot identify a proctor group 1104 that includes the same set of skills 1102 as the set of skills 1102 of the proctor profile and/or no proctor group 1104 exists that includes the same set of skills 1102 as the set of skills 1102 of the proctor profile, at step 1208, the connection system 1006 can generate a new proctor group 1104. In some embodiments, the new proctor group 1104 can include the same set of skills 1102 as the set of skills 1102 of the proctor profile.

In some embodiments, the connection system 1006 can generate a queue 1106 for the new proctor group 1104. In some embodiments, the connection system 1006 can generate a routing profile 1108 for the new proctor group 1104. In some embodiments, the connection system 1006 can include the queue 1106 for the new proctor group 1104 in the routing profile 1108.

In some embodiments, the connection system 1006 can search for other proctor groups 1104 that include a set of skills 1102 wherein the set of skills 1102 of the new proctor group 1104 includes every skill 1103 of the set of skills 1102 of the other proctor groups 1104. In some embodiments, the connection system 1006 can include each queue 1106 of the other proctor groups 1104 that include a set of skills 1102 wherein the set of skills 1102 of the new proctor group 1104 includes every skill 1103 of the set of skills 1102 of the other proctor groups 1104.

FIG. 12B shows a flowchart of a method 1250 for adding a user 1003 to a queue 1106. In some embodiments, at step 1252, the user 1003 can login to the management system 1000, the conferencing system 1002 and/or a telehealth platform. In some embodiments, the management system 1000, the conferencing system 1002, and/or the telehealth platform can include a user profile. In some embodiments, the user profile can include user information. In some embodiments, the user information can include user identity information, such as, for example, a name of the user 1003, an age of the user 1003, an identification number of the user 1003, an insurance of the user 1003, and/or any other information associated with the user 1003.

In some embodiments, the user 1003 can request a set of skills 1102. In some embodiments, if the user 1003 has previously logged into the management system 1000, the conferencing system 1002 and/or the telehealth platform, the user profile can include a set of skills 1102 previously requested by the user. In some embodiments, the management system 1000, the conferencing system 1002 and/or the telehealth platform can prompt the user 1003 to update and/or review the set of skills 1102 of the user profile. In some embodiments, the set of skills 1102 requested by the user 1003 can include one or more skills 1103 the user 1003 wants a proctor 1004 to have and/or one or more skills 1103 the proctor 1004 must have to proctor a test for the user 1003. In some embodiments, the set of skills 1102 requested by the user 1003 can include any of the skills 1103 as described above with reference to FIG. 12A.

In some embodiments, at step 1254, the management system 1000, the conferencing system 1002, the connection system 1006, and/or the telehealth platform can search for a queue 1106 for the user 1003. In some embodiments, the management system 1000, the conferencing system 1002, the connection system 1006, and/or the telehealth platform can search for a queue 1106 of a proctor group 1104 with a set of skills 1102 that includes the same skills 1103 as the set of skills 1102 requested by the user 1003.

In some embodiments, at step 1256, if the management system 1000, the conferencing system 1002, the connection system 1006, and/or the telehealth platform finds a queue 1106 of a proctor group 1104 with a set of skills 1102 that includes the same skills 1103 as the set of skills 1102 requested by the user 1003, the management system 1000, the conferencing system 1002, the connection system 1006, and/or the telehealth platform can add the user 1003 to the queue 1106.

In some embodiments, at step 1258, if the management system 1000, the conferencing system 1002, the connection system 1006, and/or the telehealth platform cannot find a queue 1106 of a proctor group 1104 with a set of skills 1102 that includes the same skills 1103 as the set of skills 1102 requested by the user 1003, the management system 1000, the conferencing system 1002, the connection system 1006, and/or the telehealth platform can search for one or more queues 1106 of a proctor group 1104 that include a set of skills 1102 that include every skill 1103 of the set of skills 1102 requested by the user 1003. In some embodiments, if the management system 1000, the conferencing system 1002, the connection system 1006, and/or the telehealth platform finds a queue 1106 that includes a set of skills 1102 that includes every skill 1103 of the set of skills 1102 requested by the user 1003, the management system 1000, the conferencing system 1002, the connection system 1006, and/or the telehealth platform can add the user 1003 to the queue 1106.

In some embodiments, if the management system 1000, the conferencing system 1002, the connection system 1006, and/or the telehealth platform finds two or more queues 1106 of proctor groups 1104 that include a set of skills 1102 that include every skill 1103 of the set of skills 1102 requested by the user 1003, the management system 1000, the conferencing system 1002, the connection system 1006, and/or the telehealth platform can add the user 1003 to an optimal queue 1106. In some embodiments, the optimal queue 1106 can include a queue 1106 of a proctor group 1104 that includes a set of skills 1102 that includes a least number of skills 1103 that are not in the set of skills 1102 requested by the user 1003. For example, in some embodiments, if the set of skills 1102 requested by the user 1003 includes a first skill 1103 and a second skill 1103, and a set of skills 1102 of a first proctor group 1104 includes the first skill 1103, the second skill 1103, and a third skill 1103, and a set of skills 1102 of a second proctor group 1104 includes the first skills 1103, the second skill 1103, the third skill 1103, and a fourth skill 1103, the management system 1000, the conferencing system 1002, the connection system 1006, and/or the telehealth platform can add the user 1003 to a queue 1106 of the first proctor group 1104.

In some embodiments, the optimal queue 1106 can include a queue 1106 with a shortest wait time. In some embodiments, the optimal queue 1106 can include a queue 1106 with the most proctors 1004 available. In some embodiments, the optimal queue 1106 can include a queue 1106 with the most total proctors 1004 available in proctor groups 1104 that include routing profiles 1108 that include the queue 1106.

In some embodiments, the optimal queue 1106 can include a queue 1106 with a highest user selected priority. In some embodiments, the management system 1000, the conferencing system 1002, the connection system 1006, and/or the telehealth platform can display a priority selection to the user 1003. In some embodiments, the priority selection can be displayed to the user 1003 if the management system 1000, the conferencing system 1002, the connection system 1006, and/or the telehealth platform finds two or more queues 1106 of proctor groups 1104 that include a set of skills 1102 that include every skill 1103 of the set of skills 1102 requested by the user 1003. In some embodiments, the priority selection can be displayed to the user 1003 if the management system 1000, the conferencing system 1002, the connection system 1006, and/or the telehealth platform finds multiple queues 1003 with a same number of skills 1103 that are not in the set of skills 1102 requested by the user 1003. In some embodiments, the priority selection can be displayed to the user 1003 if the management system 1000, the conferencing system 1002, the connection system 1006, and/or the telehealth platform is unable to determine an optimal queue. In some embodiments, the priority selection can be displayed to the user 1003 when the user 1003 requests a set of skills 1102 at step 1252.

In some embodiments, the priority selection can include an option for the user 1003 to select a skill 1103 and/or a queue parameter. In some embodiments, the queue parameter can include wait time, a number of proctors 1004 available, a number of proctor groups 1104 that include routing profiles 1108 that include a queue, or the like. For example, a user 1003 may be bilingual, and the user 1003 may select an option to prioritize a shorter wait time over testing in a particular language, and/or the user 1003 can choose to an option to prioritize a particular language over an amount of experience of a proctor 1004.

In some embodiments, at step 1256, the management system 1000, the conferencing system 1002, the connection system 1006, and/or the telehealth platform can automatically and dynamically add the user 1003 to the optimal queue 1106.

In some embodiments, the optimal queue 1106 of the user 1003 can change over time. In some embodiments, the management system 1000, the conferencing system 1002, the connection system 1006, and/or the telehealth platform can automatically and dynamically move the user 1003 to a second queue 1106 if the optimal queue 1106 changes from a first queue 1106 to the second queue 1106.

In some embodiments, a queue 1106 can include a name. In some embodiments, the name can include a maximum length of 127 characters. In some embodiments, the queue 1106 can include a description. In some embodiments, the description can include a set of skills 1103 associated with the queue 1106. In some embodiments, the description can include a maximum length of 250 characters. In some embodiments, a routing profile 1108 can include a name. In some embodiments, the name of the routing profile 1108 can include a maximum length of 127 characters. In some embodiments, the routing profile 1108 can include a description. In some embodiments, the description can include a set of skills associated with the routing profile 1108. In some embodiments, the name of the routing profile can include a maximum length of 250 characters. In some embodiments, the name and/or the description of the queue 1106 and/or the routing profile 1108 can be recalled from the memory and displayed to a proctor 1004 and/or a user 1003.

In some embodiments, the name and/or the description of a queue 1106 and/or a routing profile 1108 can be stored in a database. In some embodiments, the database can include the storage system. In some embodiments, the management system 1000, the conferencing system 1002, the connection system 1006, and/or the telehealth platform can use a hash algorithm to store and/or recall the name and/or description of the queue 1106 and/or the routing profile 1108.

### Computer Systems

FIG. 6 is a block diagram depicting an embodiment of a computer hardware system configured to run software for implementing one or more embodiments of the health testing and diagnostic systems and quality assurance, methods, and devices disclosed herein.

The example computer system 602 is in communication with one or more computing systems 620 and/or one or more data sources 622 via one or more networks 618. While FIG. 6 illustrates an embodiment of a computing system 602, it is recognized that the functionality provided for in the components and modules of computer system 602 may be combined into fewer components and modules, or further separated into additional components and modules.

The computer system 602 can comprise a module 614 that carries out the functions, methods, acts, and/or processes described herein. The module 614 is executed on the computer system 602 by a central processing unit 606 discussed further below. In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 602 includes one or more processing units (CPU) 606, which may comprise a microprocessor. The computer system 602 further includes a physical memory 610, such as random-access memory (RAM) for temporary storage of information, a read only memory (ROM) for permanent storage of information, and a mass storage device 604, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 602 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 602 includes one or more input/output (I/O) devices and interfaces 612, such as a keyboard, mouse, touch pad, and printer. The I/O devices and interfaces 612 can include one or more display devices, such as a monitor, that allows the visual presentation of data to a user. More particularly, a display device provides for the presentation of GUIs as application software data, and multi-media presentations, for example. The I/O devices and interfaces 612 can also provide a communications interface to various external devices. The computer system 602 may comprise one or more multi-media devices 608, such as speakers, video cards, graphics accelerators, and microphones, for example.

The computer system 602 may run on a variety of computing devices, such as a server, a Windows server, a Structure Query Language server, a Unix Server, a personal computer, a laptop computer, and so forth. In other embodiments, the computer system 602 may run on a cluster computer system, a mainframe computer system and/or other computing system suitable for controlling and/or communicating with large databases, performing high volume transaction processing, and generating reports from large databases. The computing system 602 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 602 illustrated in FIG. 6 is coupled to a network 618, such as a LAN, WAN, or the Internet via a communication link 616 (wired, wireless, or a combination thereof). Network 618 communicates with various computing devices 620 and/or other portable electronic devices 615. Network 618 is communicating with one or more computing systems 620 and one or more data sources 622. The module 614 may access or may be accessed by computing systems 620 and/or data sources 622 through a web-enabled user access point. Connections may be a direct physical connection, a virtual connection, and other connection type. The web-enabled user access point may comprise a browser module that uses text, graphics, audio, video, and other media to present data and to allow interaction with data via the network 618.

Access to the module 614 of the computer system 602 by computing systems 620 and/or by data sources 622 may be through a web-enabled user access point such as the computing systems' 620 or data source's 622 personal computer, cellular phone, smartphone, laptop, tablet computer, e-reader device, audio player, or another device capable of connecting to the network 618. Such a device may have a browser module that is implemented as a module that uses text, graphics, audio, video, and other media to present data and to allow interaction with data via the network 618.

The output module may be implemented as a combination of an all-points addressable display such as a cathode ray tube (CRT), a liquid crystal display (LCD), a plasma display, or other types and/or combinations of displays. The output module may be implemented to communicate with input devices 612 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 602 may comprise a physical or logical connection established between a remote microprocessor and a mainframe host computer for the express purpose of uploading, downloading, or viewing interactive data and databases online in real time. The remote microprocessor may be operated by an entity operating the computer system 602, including the client server systems or the main server system, an/or may be operated by one or more of the data sources 622 and/or one or more of the computing systems 620. In some embodiments, terminal emulation software may be used on the microprocessor for participating in the micro-mainframe link.

In some embodiments, computing systems 620 who are internal to an entity operating the computer system 602 may access the module 614 internally as an application or process run by the CPU 606.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 602 may include one or more internal and/or external data sources (for example, data sources 622). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 602 may also access one or more databases 622. The databases 622 may be stored in a database or data repository. The computer system 602 may access the one or more databases 622 through a network 618 or may directly access the database or data repository through I/O devices and interfaces 612. The data repository storing the one or more databases 622 may reside within the computer system 602.

### Additional Embodiments

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination.

Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A computer-implemented method for matching users with proctors for a proctored examination platform for medical diagnostic tests, the computer-implemented method comprising:
generating, by a computer system, one or more proctor groups, wherein each of the one or more proctor groups comprises one or more proctors, and wherein each of the one or more proctors of a proctor group comprises a set of skills, wherein each set of skills comprises one or more skills;
generating, by the computer system, a routing profile for each of the one or more proctor groups;
generating, by the computer system, a queue for each of the one or more proctor groups;
assigning, by the computer system, the queue for each of the one or more proctor groups to the routing profile of each proctor group;
assigning, by the computer system, to the routing profile of each of the one or more proctor groups, the queues of other proctor groups, wherein the set of skills of each of the one or more proctor groups comprise each of the one or more skills of the set of skills of the other proctor groups;
receiving, by a computer system, a request from a user for a proctored examination of a medical diagnostic test, wherein the request comprises a requested set of skills comprising one or more requested skills;
if a proctor group of the one or more proctor groups comprises a set of skills, wherein the set of skills is a same set of skills as the requested set of skills, assigning, by the computer system, the user to the queue of the proctor group;
if no proctor group of the one or more proctor groups comprises a set of skills, wherein the set of skills is the same set of skills as the requested set of skills, assigning, by the computer system, the user to a queue of a proctor group with a highest priority, wherein the set of skills of the proctor group comprises every requested skill of the requested set of skills;
establishing, by the computer system, a video conferencing session between a user device of the user and a proctor device of a proctor, wherein the proctor is an available proctor of any of the proctor groups comprising a routing profile that comprises the queue of the user.

2. The computer-implemented method of claim 1, wherein the one or more skills of each set of skills comprise a language, a step of the medical diagnostic test, a location, and/or a test type.

3. The computer-implemented method of claim 1, wherein the proctor group with the highest priority is determined by the computer system based on a priority selection of the user.

4. The computer-implemented method of claim 1, wherein the queues of each routing profile are ranked based on a priority level of the queues.

5. The computer-implemented method of claim 4, wherein the queue with a top priority level is the queue of the proctor group that comprises the routing profile.

6. The computer-implemented method of claim 4, wherein the priority level of the queues is based on a priority level of one or more of the skills of the set of skills of the proctor group of the queue.

7. The computer-implemented method of claim 4, wherein the priority level of the queues is based on a similarity of the set of the skills of the proctor group of that comprises the routing profile and the set of skills of the proctor group of the queue.

8. The computer-implemented method of claim 1, wherein the proctor is a first proctor, wherein the first proctor observes, via the video conferencing session, the user performing a first step of the medical diagnostic test, and wherein the computer-implemented method further comprises the step of:
after the first step of the medical diagnostic test is completed by the user, connecting, by the computer system, a second proctor to the video conferencing session, to observe the user performing the second step of the medical diagnostic test.

9. The computer-implemented method of claim 8, wherein the computer system is configured to display a computer generated representation of a proctor to the user via the user device, and wherein the computer generated representation of the proctor is a same computer generated representation for the first proctor and the second proctor.

10. A computer-implemented method for matching users with proctor for a proctored examination platform for medical diagnostic tests, the computer-implemented method comprising:
generating, by a computer system, a proctor group, wherein the proctor group comprises one or more proctors, and wherein each of the one or more proctors of the proctor group comprises a set of skills, wherein the set of skills comprises one or more skills;
generating, by the computer system, a routing profile for the proctor group;
generating, by the computer system, a queue for each of the one or more proctor groups;
assigning, by the computer system, the queue to the routing profile;
assigning, by the computer system, to the routing profile, queues of other proctor groups, wherein the set of skills of the proctor group comprises every skill of a set of skills of the other proctor groups;
ranking, by the computer system, each queue of the routing profile based on a priority level of each queue, wherein a queue with a top priority level is the queue of the proctor group, and wherein a priority level the queues of the other proctor groups is based on a number of skills of the set of skills of the proctor group of the routing profile not in the set of skills of the other proctor groups;
establishing, by the computer system, a video conferencing session between an available proctor of the proctor group and a highest priority user, wherein the highest priority user comprises a user in a queue with a highest priority.

11. The computer-implemented method of claim 10, wherein the one or more skills of the set of skills of the proctor group comprise a language, a step of the medical diagnostic test, a location, and/or a test type.

12. The computer-implemented method of claim 10, wherein the queue with the highest priority is a non-empty queue with a highest-priority level.

13. A computer-implemented system for matching users with proctors for a proctored examination platform for medical diagnostic tests, the computer-implemented system comprising:
at least one processor in communication with at least one memory storing instructions that cause the at least one processor to implement the proctored examination platform by:
generating, by the computer-implemented system, one or more proctor groups, wherein each of the one or more proctor groups comprises one or more proctors, and wherein each of the one or more proctors of a proctor group comprises a set of skills, wherein each set of skills comprises one or more skills;
generating, by the computer-implemented system, a routing profile for each of the one or more proctor groups;
generating, by the computer-implemented system, a queue for each of the one or more proctor groups;
assigning, by the computer-implemented system, the queue for each of the one or more proctor groups to the routing profile of each proctor group;
assigning, by the computer-implemented system, to the routing profile of each of the one or more proctor groups, the queues of other proctor groups, wherein the set of skills of each of the one or more proctor groups comprise each of the one or more skills of the set of skills of the other proctor groups;
receiving, by the computer-implemented system, a request from a user for a proctored examination of a medical diagnostic test, wherein the request comprises a requested set of skills comprising one or more requested skills;
if a proctor group of the one or more proctor groups comprises a set of skills, wherein the set of skills is a same set of skills as the requested set of skills, assigning, by the computer-implemented system, the user to the queue of the proctor group;
if no proctor group of the one or more proctor groups comprises a set of skills, wherein the set of skills is the same set of skills as the requested set of skills, assigning, by the computer-implemented system, the user to a queue of a proctor group with a highest priority, wherein the set of skills of the proctor group comprises every requested skill of the requested set of skills;
establishing, by the computer-implemented system, a video conferencing session between a user device of the user and a proctor device of a proctor, wherein the proctor is an available proctor of any of the proctor groups comprising a routing profile that comprises the queue of the user.

14. The computer-implemented system of claim 13, wherein the one or more skills of each set of skills comprise a language, a step of the medical diagnostic test, a location, and/or a test type.

15. The computer-implemented system of claim 13, wherein the proctor group with the highest priority is determined by the computer-implemented system based on a priority selection of the user.

16. The computer-implemented system of claim 13, wherein the queues of each routing profile are ranked based on a priority level of the queues.

17. The computer-implemented system of claim 16, wherein the queue with a top priority level is the queue of the proctor group that comprises the routing profile.

18. The computer-implemented system of claim 16, wherein the priority level of the queues is based on a priority level of one or more of the skills of the set of skills of the proctor group of the queue.

19. The computer-implemented system of claim 16, wherein the priority level of the queues is based on a similarity of the set of the skills of the proctor group of that comprises the routing profile and the set of skills of the proctor group of the queue.

20. The computer-implemented system of claim 13, wherein the computer-implemented system comprises at least one processor and at least one non-transitory storage medium storing instructions that are executed by the processor.
